(19) United States
(12) Patent Application Publication
(10) Pub. No.: US 2013/0130387 A1
(43) Pub. Date: May 23, 2013

(54) METHOD FOR GENERATING INDUCED PLURIPOTENT STEM CELLS FROM KERATINOCYTES DERIVED FROM PLUCKED HAIR FOLLICLES

(71) Applicants: Joseph Itskovitz-Eldor, Haifa (IL); Atara Novak-Petraro, Haifa (IL); Ronit Shtrichman, Haifa (IL)

(72) Inventors: Joseph Itskovitz-Eldor, Haifa (IL); Atara Novak-Petraro, Haifa (IL); Ronit Shtrichman, Haifa (IL)

(73) Assignee: Technion Research & Development Foundation Limited, Haifa (IL)

(21) Appl. No.: 13/812,566

(22) PCT Filed: Jul. 27, 2011
(86) PCT No.: PCT/IL11/00606

§ 371 Date: Jan. 28, 2013

Related U.S. Application Data
(60) Provisional application No. 61/367,933, filed on Jul. 27, 2010.

(51) Int. Cl. C12N 15/85

(52) U.S. Cl. CPC C12N 15/85
USPC 435/456; 435/366

(57) ABSTRACT

A method for generating induced pluripotent stem (iPS) cells from isolated hair follicles is disclosed. The method comprises:
a. culturing isolated hair follicle keratinocytes on a layer of feeder cells, so as to generate colonies of hair follicle keratinocytes;
b. detaching the colonies of hair follicle keratinocytes from the feeder cells so as to generate detached keratinocytes;
c. infecting the detached keratinocytes with a virus comprising a nucleic acid molecule encoding at least one dedifferentiation factor so as to generated infected keratinocytes; and
d. culturing the infected keratinocytes on a layer of feeder cells in a culture medium until iPS cells are formed, thereby generating iPS cells.
Populations and uses of the iPS cells are also disclosed.

### TECHNICAL FIELD

The present invention relates to the field of pluripotent stem cell generation from somatic cells and in particular from keratinocytes derived from plucked hair follicle. The invention further relates to therapeutic use of the pluripotent stem cells and to their use in drug screening and disease modeling.

### BACKGROUND ART

Induced pluripotent stem cells (iPSCs) are human somatic cells that were reprogrammed into a pluripotent state resembling that of human embryonic stem cells (hESCs). iPSCs are generated by introducing a defined set of transcription factors, including Oct4 (Octamer-4, also known as POU5F1), Sox2 (SRY (sex determining region Y)-box 2), Klf4 (Krüppel-like factor 4) and c-Myc or Nanog and Lin 28 (Takahashi et al. 2007; Yu et al. 2007). The seminal achievement of induced pluripotency holds great promise for regenerative medicine. Patient-specific iPSCs can provide useful platforms for the discovery of new drugs, as well as unprecedented insights into disease mechanisms that ultimately may be used to develop cell and tissue replacement therapies (Kiskinis and Eggan 2010).

Human iPSCs have been generated from various types of somatic cells, most commonly fibroblasts (Takahashi et al. 2007; Lowry et al. 2008; Park et al. 2008; Huangfu et al. 2008h; Soldner et al. 2009) that are isolated from tissues harvested via surgical intervention. Blood is a cell source that can be easily obtained from most patients, but a practical reprogramming protocol of human peripheral blood cells has not yet been successful. A recent study reported the reprogramming of cord blood derived endothelial cells into iPSCs (Haase et al. 2009). However, cord blood cannot be obtained directly from most patients, and is therefore an unsuitable source for modeling specific diseases.

Aasen et al. have reported the reprogramming of human primary keratinocytes derived from skin biopsy or from plucked human hair (Aasen et al, 2008). However, the reprogramming efficiency obtained with the existing methods does not allow for consistent generation of iPSCs in large numbers required for therapeutic and other uses.

### SUMMARY OF INVENTION

The findings of the present invention demonstrate an efficient and reproducible method for the derivation of iPSCs from human hair. The generated iPSCs are pluripotent and able to further differentiate into any of the three germ layers and develop into e.g. functional cardiomyoctes. Furthermore, this protocol is the most efficient method described so far for generating human iPSCs from human keratinocytes while using a single lentiviral vector for human cells and is suitable for generating experimental models of human diseases for research and clinical applications.

According to an aspect of some embodiments of the present invention there is provided a method for generating induced pluripotent stem (iPS) cells from isolated hair follicles, the method comprising:

a. culturing isolated hair follicle keratinocytes on a layer of feeder cells, so as to generate colonies of hair follicle keratinocytes;

b. detaching the colonies of hair follicle keratinocytes from the feeder cells so as to generate detached keratinocytes;

c. infecting the detached keratinocytes with a virus comprising a nucleic acid molecule encoding at least one dedifferentiation factor so as to generate infected keratinocytes; and

d. culturing the infected keratinocytes on a layer of feeder cells in a culture medium until iPS cells are formed, thereby generating iPS cells.

According to an aspect of some embodiments of the present invention there is provided induced pluripotent stem (iPS) cells obtained according to the method described herein.

According to an aspect of some embodiments of the present invention there is provided a pharmaceutical composition comprising the iPS cells of the present invention.

According to an aspect of some embodiments of the present invention there is provided a method of generating lineage specific cells, the method comprising:

(a) generating iPS cells according to the method described herein; and

(b) ex vivo differentiating the iPS cells into lineage specific cells, thereby generating the lineage specific cells.

According to some embodiments of the invention, the isolated hair follicle keratinocytes are generated by dissociating cells of the isolated hair follicles.

According to some embodiments of the invention, the colonies comprise between 20-30 hair follicle keratinocytes.

According to some embodiments of the invention, the isolated hair follicle keratinocytes are in contact with the virus for less than 2 hours.

According to some embodiments of the invention, the isolated hair follicle keratinocytes are in contact with the virus for less than one hour.

According to some embodiments of the invention, the virus is a lentivirus.

According to some embodiments of the invention, the isolated hair follicle keratinocytes are not passaged for more than 3 passages.

According to some embodiments of the invention, the isolated hair follicle keratinocytes are passaged for 2-3 passages.

According to some embodiments of the invention, the dissociating is effected using trypsin.

According to some embodiments of the invention, the infecting is effected during centrifugation at a centrifugal force of about 200 g to about 1000 g

According to some embodiments of the invention, the infecting is effected at a temperature between 25° C.-37° C.

According to some embodiments of the invention, the feeder cells comprise 3T3 cells or mouse embryonic feeder (MEF) cells.

According to some embodiments of the invention, the nucleic acid molecule further encodes LoxP sites.

According to some embodiments of the invention, the method further comprises excising the nucleic acid molecule following step (d) by contacting the iPS cells with a cre-recombinase enzyme.

According to some embodiments of the invention, the at least one dedifferentiation factor is selected from the group consisting of OCT4, SOX2, KLF4, C-MYC, Nanog and Lin 28.

According to some embodiments of the invention, the at least one dedifferentiation factor is selected from the group consisting of OCT4, SOX2, KLF4 and C-MYC.

According to some embodiments of the invention, the at least one dedifferentiation factor is OCT4, SOX2 and KLF4.

According to some embodiments of the invention, the at least one dedifferentiation factor is OCT4, SOX2 and C-MYC.

According to some embodiments of the invention, the at least one dedifferentiation factor is OCT4 and SOX2.

According to some embodiments of the invention, the nucleic acid molecule comprises a sequence as set forth in SEQ ID NO: 1.

According to some embodiments of the invention, wherein, for at least a portion of a time of the culturing the infected keratinocytes, the culture medium comprises a small molecule.

According to some embodiments of the invention, the small molecule is selected from the group consisting of a glycogen synthase kinase 3 (GSK-3) inhibitor, a lysine-specific demethylaseinhibitor, a histone methyltransferase inhibitor, a histone deacetylase inhibitor, a TGF-β inhibitor; a combination of inhibitors of mitogen-activated protein kinase kinase (MAPK/ERK kinase or MEK) and GSK-3; and an L-type calcium channel agonist.

According to some embodiments of the invention, the GSK-3 inhibitor comprises CHIR99021.

According to some embodiments of the invention, the lysine-specific demethylase inhibitor is Parnate (Tranylcypromine).

According to some embodiments of the invention, the detaching is effected using EDTA.

According to some embodiments of the invention, there is provided a cell line of the iPS cells of the present invention.

According to some embodiments of the invention, the iPS cells are used in tissue regeneration.

According to some embodiments of the invention, the tissue regeneration is cardiac tissue regeneration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-F depict derivation of hair follicle keratinocytes (HFKTs) and their characterization. A) A bulk of intact plucked hair follicles, and B) following enzymatic removal of cells, generating single cell suspension. C) isolated from the plucked hair and seeded on inactivated 3T3 feeder cells appeared as small colonies one day after passaging, and D) as large colonies 6 days after passaging. E) QRT-PCR of keratinocyte markers and F) Quantitative Real-time PCR (QRT-PCR) of the 4 reprogramming factors, was applied using RNA isolated from HFKTs, HaCat cells and hESCs (H9.2). Analysis was carried out using Glyceraldehyde 3-phosphate dehydrogenase (GAPDH) gene expression as internal control. Data are presented relative to gene expression in HaCat cells. RQ, Relative Quantification.

FIGS. 2A-D show generation and characterization of HFKT-iPSCs. A) Illustration of the HFKT reprogramming procedure. B) Morphology of HFKT-iPSC colonies derived from KTN and KTR source cells at passage 25, relative to hESC colony H9.2 at passage 29+43. C) Immunostaining of Kl4, Klf4 and c-Myc, in HFKT donor cells (KTR P.2), their derived iPSCs (HFKT-iPS) (KTR13 p. 22) and hESCs from line H9.2. Nuclei are stained with DAPI (blue). Scale bar represents 100 μm, excluding Kl4 staining of HFKT in which scale bar represents 50 μm. D) FACS analysis of ESC markers for HFKT donor cells (KTR p. 2), their derived iPSCs (HFKT-iPS) (KTR13 p. 24) and hESCs from line H9.2. Positive cells' percentages and Geome Mean (G.M) indicating signal intensity are presented. Data were obtained relative to negative control cells stained with only a secondary antibody. p2, p22, and p. 24, passage 2, 22 and 24, respectively. KTN and KTR, HFKTs obtained from the individuals “N” and “R”, respectively.

FIGS. 3A-B demonstrate pluripotency of HFKT-iPSCs. A) Immunostaining of typical hESC (H9.2) markers shown for HFKT-iPSCs clone KTR13 P.20. Nuclei are stained with DAPI (blue). Scale bar represents 100 μm. B) QRT-PCR measuring the expression levels of the reprogramming factors in undifferentiated HFKT-iPSC clones following 3 different passages: p5-7, p12-17 and p22-25. Analysis was performed using GAPDH internal control. Data are presented relative to hESC transcript levels.

FIG. 4 shows RT-PCR analysis of pluripotent genes. RT-PCR was performed to the following samples: hESCs—H9.2, HFKTs, HFKT-iPSCs clones KTN5, KTN7, KTR12 and KTR13, using primers flanking the pluripotent genes: Oct4, Sox2, Nanog, Rex1, c-Myc and Klf4. GAPDH was used as internal control. The primers list is described in Table 1.

FIGS. 5A-J show differentiation of HFKT-iPSCs. A) Five day-old embryoid bodies (EBs) derived from HFKT-iPSC KTR13 clone. Similar morphologies were obtained for all HFKT-iPSC clones. B-F) Immunostaining of 21 day-old EBs derived from KTR13 clone revealed expression of mesodermal (SMA—B, CD31—C), ectodermal (tubulinβ3, Nestin—D) and endodermal (AFP—E, Glucagon—F) marker proteins. Nuclei are stained with DAPI (blue). Scale bar represents 100 μm, excluding tubulinβ3 and Nestin staining, in which scale bar represents 20 μm. G-I) Teratoma formation obtained from HFKT-iPSC KTR13 clone. G) Neuronal tissue represents ectodermal lineage. H) Endodermal epithelium with prominent mucus-producing cells representing endoderm formation. I) Adipose and muscle tissues as well as chondrocyte area pointing to mesoderm formation. Scale bar represents 50 μm. J) QRT-PCR measuring the expression of the reprogramming factors following differentiation. Analysis was carried out using GAPDH internal control. Data are presented relative to hESC (H9.2) transcript levels. SMA, smooth muscle actin: AFP, endodermal alpha-fitoprotein.

FIGS. 6A-E depict cardiac differentiation of HFKT-iPSCs. A) Immunofluorescence staining of cardiac proteins in iPSC-derived cardiomyocytes (CMs). Micro-dissected contracting areas from HFKT-iPSC-CMs were stained for typical myofilament proteins. Cells (clone KTR13, 36 day-old EBs) were co-labeled with anti-cardiac troponin I (green) and either anti-sarcomeric α-actinin or myosin heavy chain (MHC, red). Nuclei were stained with DAPI (blue). A representative area with apparent cross-striations is focused in the insert. B) Extracellular electrograms recorded by means of the Microelectrode Array (MEA) data acquisition system. A spontaneously contracting EB (clone KTR13, 24 day-old EB) was seeded over the recording electrodes (left panel). A representative display of electrogram recorded from the MEA array (middle panel) and a representative analog recording from electrode #47 (right panel), are shown. C) Representative action potential recordings from a spontaneously contracting EB (clone KTR13, 37 day-old EB), demonstrating the pacemaker activity of the HFKT-iPSC-CMs. The right panel is an expanded time scale taken from the region indicated with a dot on the left panel. D-E) Simultaneous recordings of [Ca²⁺]ᵢ transients (D) and contraction (E) of HFKT-iPSC-CMs and the effect of isoproterenol in a representative experiment (clone KTR13, 37 day-old EB) illustrating the increase in contraction amplitude in response to increase of isoproterenol concentrations. Control, vehicle.

FIGS. 7A-C demonstrate Cre-mediated excision of the loxP-containing polycistronic lentiviral STEMCCA vector (see Material and Methods): A) Genomic PCR, verifying the excision of the lentiviral vector from 2 HFKT-iPSC clones KTR13 and KTN7, was done using two primer sets flanking the WPRE region of the lentiviral vector. Lanes 1-5 show PCR products of 173 bp, obtained with WPRE 9142-9633 primer set. Lanes 6-10 show PCR products of 491 bp, obtained with WPRE 9142-9633 primer set. Lanes 1 and 6: Cre-KTN7.3 excised HFKT-iPSC clone. Lanes 2 and 7: Cre-KTR13.4 excised HFKT-iPSC clone. Lanes 3 and 8: KTN7 HFKT-iPSC clone. Lanes 4 and 9: KTR13 HFKT-iPSC clone. Lanes 5 and 10: No DNA—negative control. B) QRT-PCR measuring the expression of the reprogramming factors in the excised Cre-HFKT-iPSC clones. Analysis was done using GAPDH internal control. Data are presented relative to hESC transcript levels. RQ, Relative Quantification. C) Table.

FIG. 8 shows morphology and immunostaining of typical hESC markers; Oct4, Sox2, SSEA4, Tral-60 and Tral-81 shown for excised HFKT-iPSCs clone Cre-KTN7.3 P.15+33. Nuclei are stained with DAPI (blue). Scale bar represents 100 μm.

FIGS. 9A-F shows morphology of 7 day-old EBs (A) and immunostaining of 21 day-old EBs derived from excised HFKT-iPSC clone Cre-KTN7.3 P. 15+27, revealed expression of mesodermal (SMA—B, CD31—C), ectodermal (tubulinβ3, Nestin—D) and endodermal (AFP—E, Glucagon—F) marker proteins. Nuclei are stained with DAPI (blue). Scale bar represents 100 μm.

### DETAILED DESCRIPTION OF THE INVENTION

The present invention relates to the field of pluripotent stem cell generation from somatic cells and in particular from keratinocytes derived from plucked hair follicle. The invention further relates to therapeutic use of the pluripotent stem cells and to their use in drug screening and disease modeling.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Plucked hair follicles have been used as a convenient sample material for studying genetic disorders and for diagnostic purposes. Hair follicles also provide an interesting model system of epithelial cells for biomedical research (Limat et al. 1986).

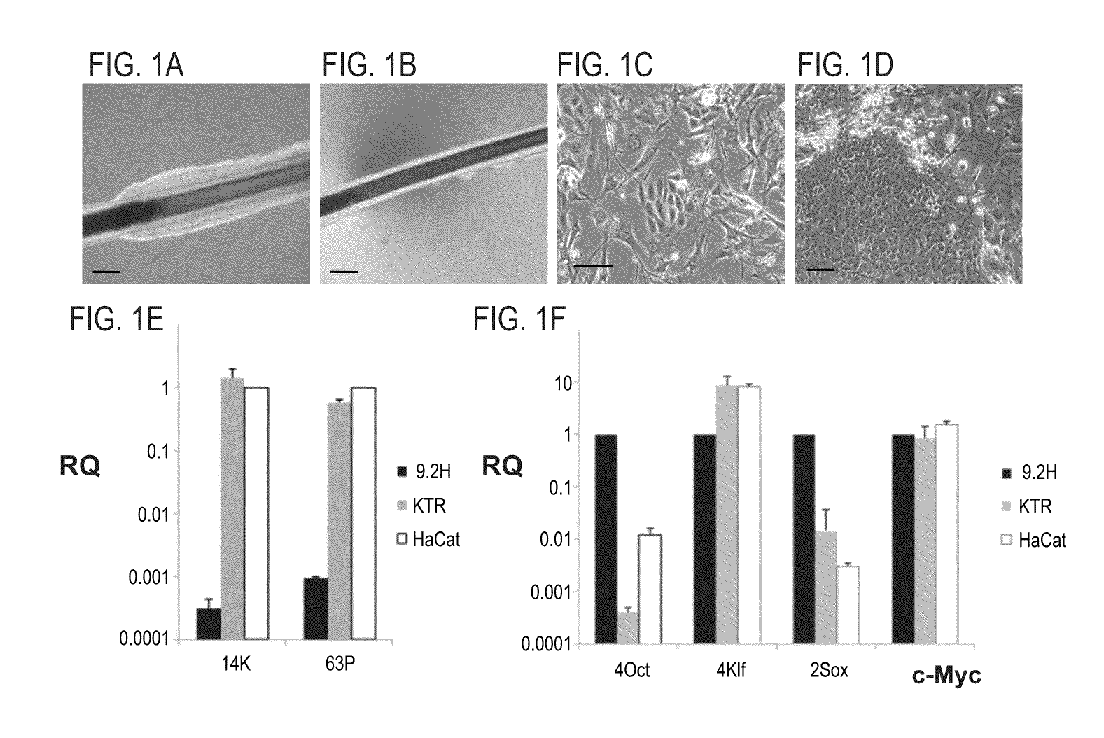

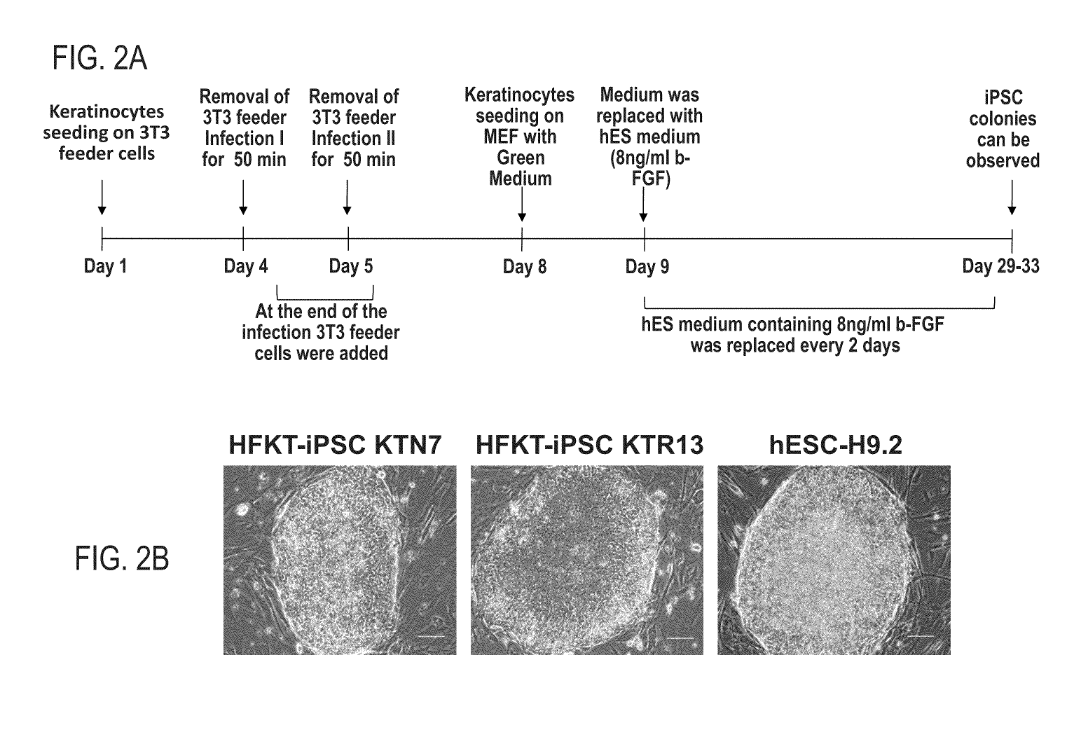

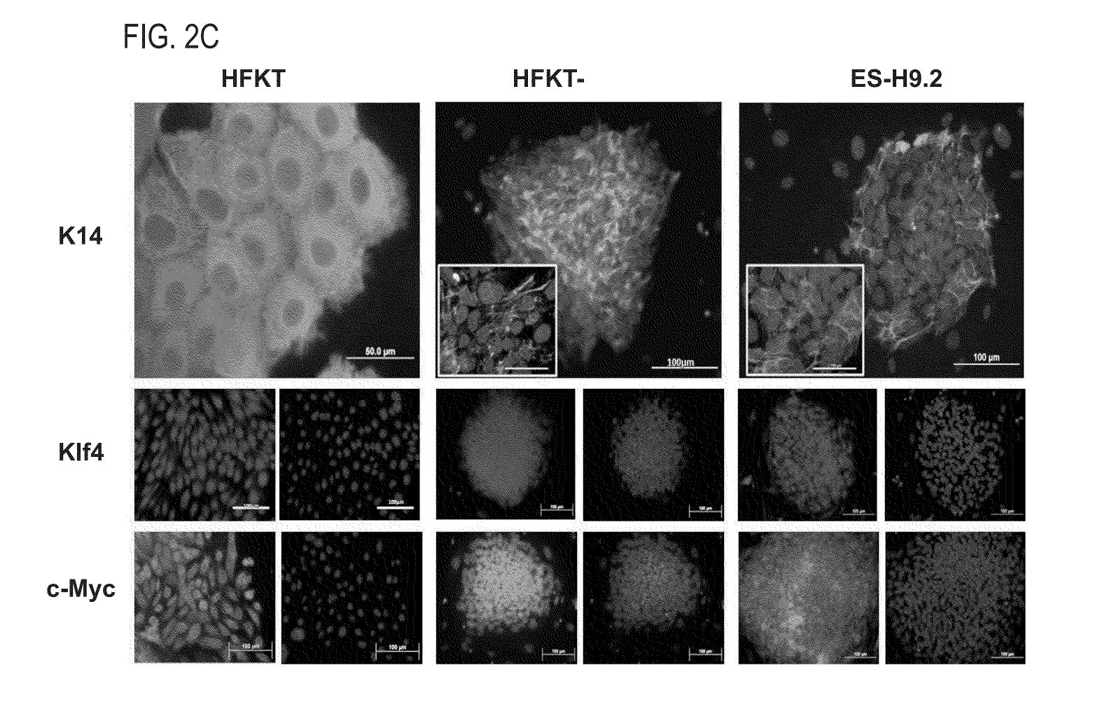

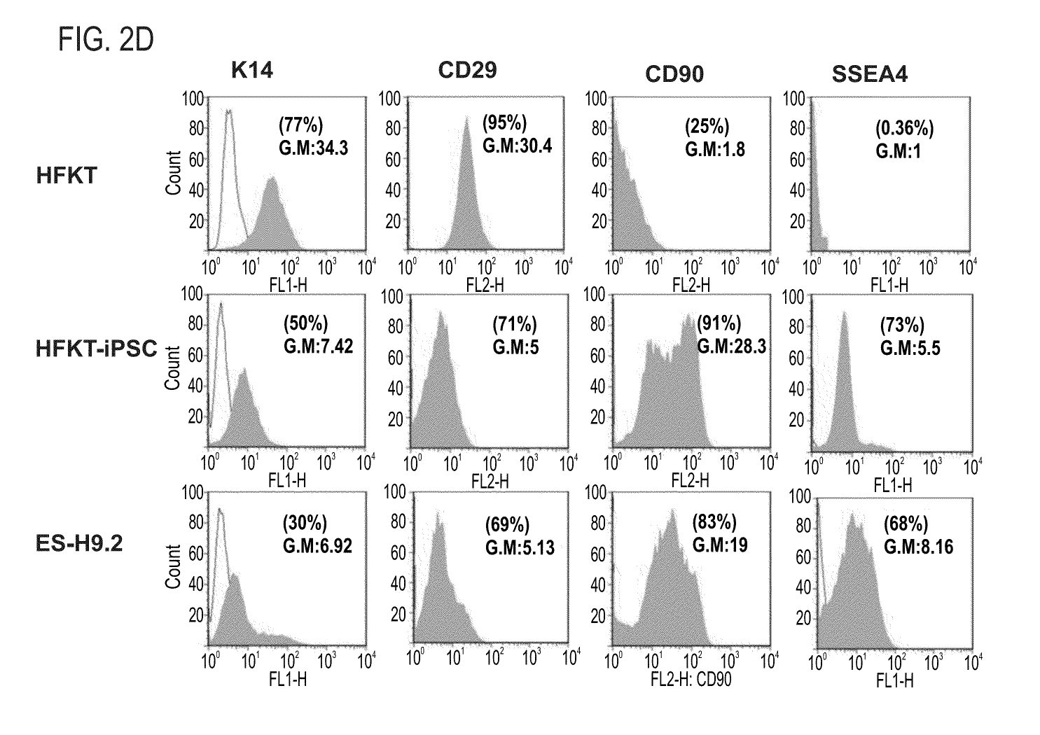

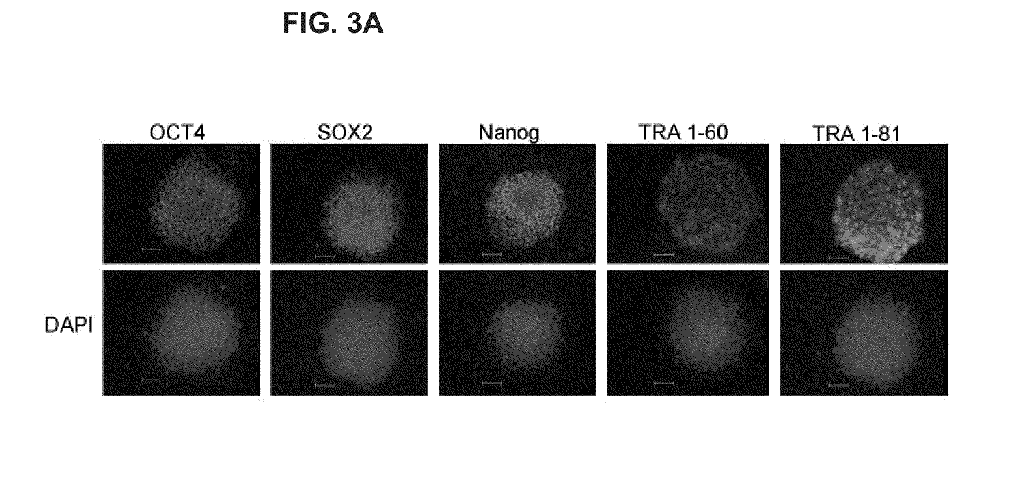

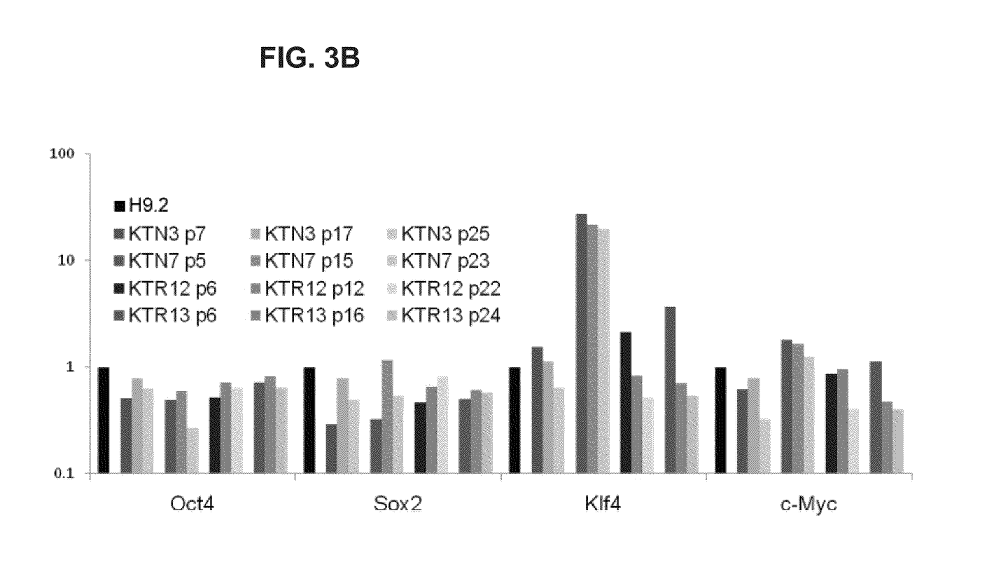

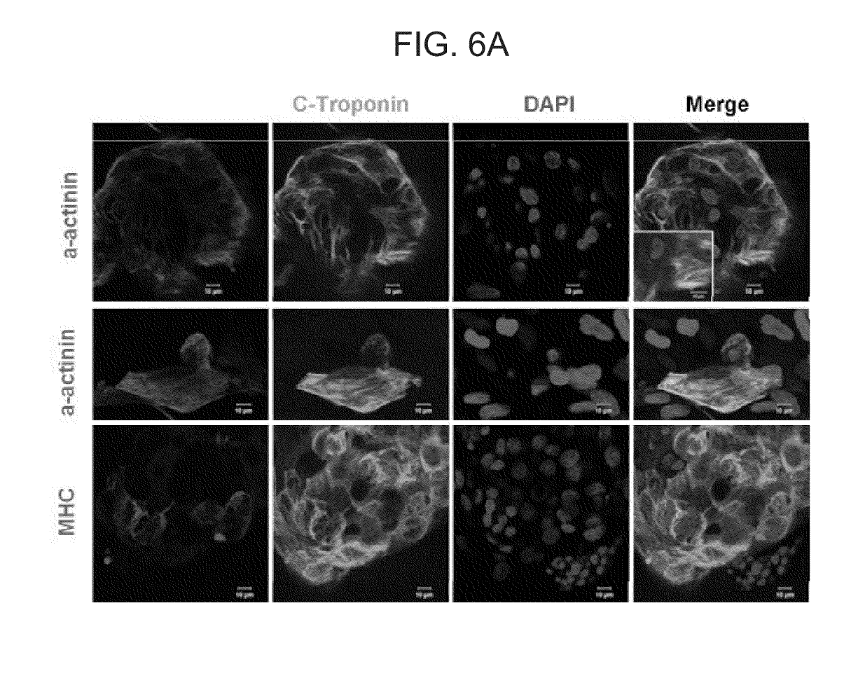

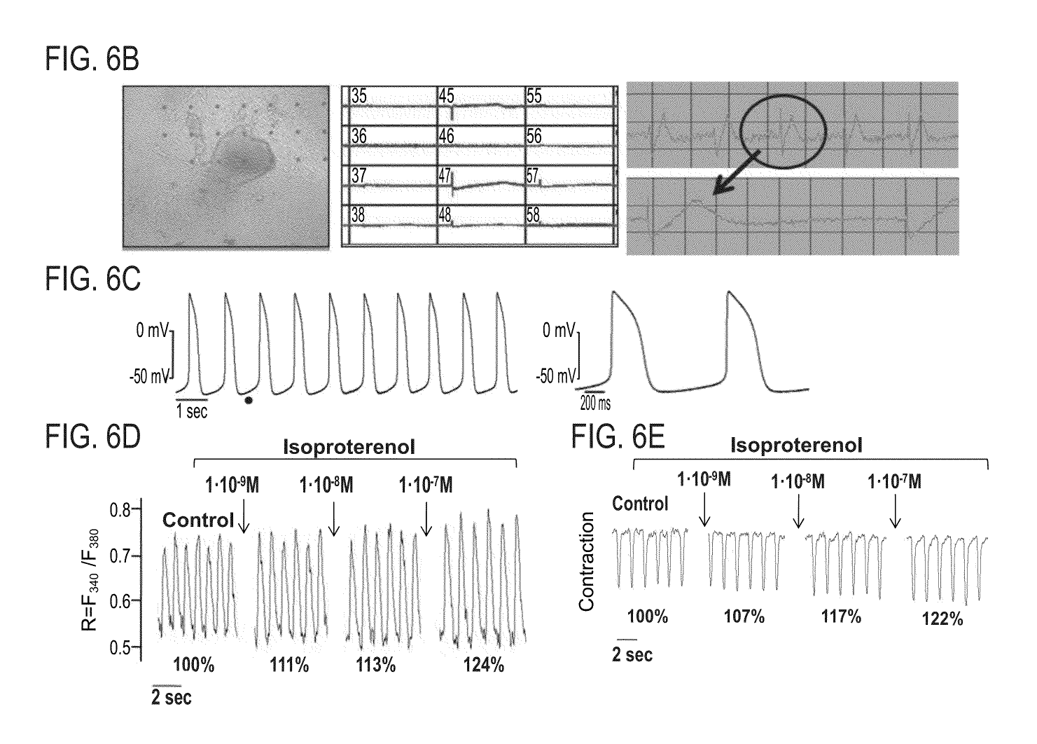

Due to the low efficiency of generating iPS cells from adult human hair according to published protocols, the present inventors have devised novel methods for the generation of such cells. Such methods include the isolation of keratinocytes from the outer root sheath of plucked hair and growing the cells on feeder cells in an appropriate medium. The present inventors have further found that prior to the infection stage of the protocol, it is necessary to remove the feeder cells and provide them again immediately following the infection. Successful generation of iPS cells from isolated human keratinocytes is demonstrated in FIGS. 1A-D and FIGS. 2A-D. The present inventors demonstrated the pluripotency of the generated cells using several assays (FIGS. 3A-B, 4, 5A-J, 8 and 9A-F) and further showed that the cells could be ex-vivo differentiated towards a cardiac lineage (FIGS. 6A-E),

Thus, according to one aspect of the present invention there is provided a method for generating induced pluripotent stem (iPS) cells from isolated hair follicles, the method comprising:

a. culturing isolated hair follicle keratinocytes on a layer of feeder cells, so as to generate colonies of hair follicle keratinocytes;

b. detaching the colonies of hair follicle keratinocytes from the feeder cells so as to generate detached keratinocytes;

c. infecting the detached keratinocytes with a virus comprising a nucleic acid molecule encoding at least one dedifferentiation factor so as to generate infected keratinocytes; and

d. culturing the infected keratinocytes on a layer of feeder cells in a culture medium until iPS cells are formed, thereby generating iPS cells.

As used herein, the term “pluripotent cell” refers to a cell that has the potential to divide in vitro for a long period of time (e.g., greater than one year) and has the unique ability to differentiate into cells derived from all three embryonic germ layers—endoderm, mesoderm and ectoderm. Pluripotent cells have the potential to differentiate into the full range of daughter cells having distinctly different morphological, cytological or functional phenotypes unique to different specific tissues. By contrast, descendants of pluripotent cells are progressively restricted in their differentiation potential, with some cells eventually having only one fate.

The phrase “isolated hair follicle” refers to the hair follicle removed from the attached outer hair. The hair follicle comprises concentric layers containing the inner sections of the hair shafts, surrounded by the inner root sheath (IRS). The IRS is composed of terminally differentiated keratinocytes and is encircled by the outer root sheath (ORS) which is the outermost layer of the hair follicle.

The phrase “isolated hair follicle keratinocytes” refers to a population of keratinocytes obtained from the hair follicle wherein the population is devoid of at least a portion of other hair follicle cells, such as those found in the arrector pili muscles, sebaceous glands and apocrine sweat glands.

According to one embodiment, the population of hair follicle keratinocytes is a pure (at least 90% pure) population of hair follicle keratinocytes.

The hair follicle keratinocytes may be derived from any mammal e.g. humans.

According to one embodiment, the keratinocytes are separated from other hair follicle cells using a dispersing agent including, but not limited to trypsin. Mechanical dispersion is also contemplated. A combination of mechanical dispersion and trypsinization may lead to the generation of a single cell suspension.

Trypsinization may be performed according to any protocol known in the art, for example, but not limited to, by incubation with about 0.1% Trypsin and about 0.02% EDTA.

As mentioned, the isolated hair follicle keratinocytes are cultured on feeder cells under conditions that allow generation of hair follicle keratinocytes colonies.

Thus, for example the keratinocytes may be seeded in one, two or three wells of a 6-well plate (on a layer of feeder cells), depending on the amount of keratinocytes obtained. A 6-well plate is commonly used in the art of cell culturing and has the outer dimensions of a standard micro plate. One well in a 6 well-plate has a growth area of about 9.6 cm² and a working volume of about 2 ml to about 5 ml.

Contemplated growth media for culturing the keratinocytes include, but are not limited to DMEM and/or DMEMF12. The medium may or may not comprise animal serum. According to one embodiment, the medium comprises epidermal growth factor. According to a particular embodiment, the medium is Green medium (60% DMEM, 30% DMEM F-12, 10% Fetal Bovine Serum, 1 mM Sodium Pyruvate, 2 mM L-Glutamine, 5 μg insulin, 0.5 μg/ml Hydrocortisone, 0.2 nM Adenine, 2 nM triiodothyronine (T3), 10 ng/ml Epidermal Growth Factor and 100 U/ml penicillin, 100 μg/ml streptomycin).

The particular type of feeder cells used to support the growth of the keratinocytes depends on the growth conditions and may be chosen from, without being limited to, NIH-3T3 cells (ATTC CRL-1658), J2 or Jmax-3T3 cells (see Pellegrini et al., 2001, Pa-6 cells (Riken Bioresource Center Cell Bank, Koyadai, Japan, Cat. No. RCB1127), or fibroblasts cells such as HFF (ATTC SCRC-1041) or HDF. Optionally, the cells may be cultured on a cell-free matrix such as gelatin, or collagen.

According to a specific embodiment, the isolated keratinocytes are cultured on 3T3 feeder cells.

It is common practice to inactivate the feeder cells prior to use, i.e. to manipulate the feeder cells to prevent them from reproducing, for example by exposing them to a chemical agent, such as Mitomycin C, that inhibits DNA synthesis and consequently prevents cytokinesis. In certain embodiments, the feeder cells used for the culturing of isolated keratinocytes comprise inactivated MEF feeder cells, as defined herein below in “Materials and Methods”.

According to another embodiment, the keratinocytes have not been frozen (i.e. freshly isolated).

In certain embodiments, the keratinocyte density that is optimal for lentivirus infection is obtained by seeding the keratinocytes at a cell density selected from a range of about 10000-60000 and 20000-40000, preferably 30000 cells/well of a 6-well plate, i.e. at a cell density selected from a range of about 1000-6000 and 2000-4000, preferably 3000 cells/cm².

Preferably, the keratinocytes are not passaged for more than 4 passages prior to infection.

More preferably, the keratinocytes are not passaged for more than 3 passages prior to infection.

According to a particular embodiment, the keratinocytes are passaged for 2-3 passages prior to infection.

Once colonies of about 20-30 keratinocytes are formed, they are detached from the feeder cells.

This may be effected according to any method known in the art including for example, incubation of the cells in EDTA (at a concentration ranging from about 0.01% to about 0.02%) for no more than 8 minutes at 37° C. until the feeder cells are visibly detached and the keratinocytes still adhere to the plate.

In order to generate iPS cells from the keratinocytes, dedifferentiating factors are expressed in the keratinocytes as described herein below.

Exemplary dedifferentiating factors include, but are not limited to OCT4, SOX2, KLF4, C-MYC, Nanog and Lin 28.

Typically, at least four dedifferentiating factors are expressed in the cells, however the number of dedifferentiating factors may be reduced if following introduction into the hair follicle keratinocytes, the transduced cells are incubated in a medium comprising molecules which alter transduction pathways and/or chromatin, as further described herein below.

According to one embodiment the method is effected by expressing in the cells at least one polypeptide belonging to the Oct family or the Sox family.

According to another embodiment, the method is effected by expressing in the cells at least two polypeptides—one belonging to the Oct family and one to the Sox family.

Examples of polypeptides belonging to the Oct family include, for example, Oct3/4 (NM_{—}013633, mouse and NM_{—}002701, human), Oct1A (NM_{—}198934, mouse and NM_{—}002697, human), Oct6 (NM_{—}011141, mouse and NM_{—}002699, human), and the like. Oct3/4 is a transcription factor belonging to the POU family, and is reported as a marker of undifferentiated cells (Okamoto et al., Cell 60:461-72, 1990). Oct3/4 is also reported to participate in the maintenance of pluripotency (Nichols et al., Cell 95:379-91, 1998).

Examples of polypeptides belonging to the Sox (SRY-box containing) family include, for example Sox1 (NM_{—}009233, mouse and NM_{—}005986, human), Sox3 (NM_{—}009237, mouse and NM_{—}005634, human), Sox7 (NM_{—}011446, mouse and NM_{—}031439, human), Sox15 (NM_{—}009235, mouse and NM_{—}006942, human), Sox17 (NM_{—}011441, mouse and NM_{—}022454, human) and Sox18 (NM_{—}009236, mouse and NM_{—}018419, human), and a preferred example includes Sox2 (NM_{—}011443, mouse and NM_{—}003106, human).

According to yet another embodiment, the method is effected by expressing in the cells four polypeptides—one belonging to the Oct family, one belonging to the Sox family, Nanog and lin28.

Alternatively, the method is effected by expressing in the cells four polypeptides—one belonging to the Oct family, one belonging to the Sox family, Klf-4 and c-Myc.

Expressing the dedifferentiating factors described herein above in the keratinocytes may be performed by genetic manipulation—example using expression constructs. Various methods can be used to introduce the expression vectors of the present invention into the hair follicle keratinocytes. Such methods are generally described in, for instance: Sambrook, J. and Russell, D. W. (1989, 1992, 2001), Molecular Cloning: A Laboratory Manual, Cold Springs Harbor Laboratory, New York; Ausubel, R. M. et al., eds. (1994, 1989). Current Protocols in Molecular Biology, John Wiley and Sons, Baltimore, Md. (1989); Chang, P. L., ed. (1995). Somatic Gene Therapy, CRC Press, Boca Raton, Fla.; Vega, M. A. (1995). Gene Targeting, CRC Press, Boca Raton, Fla.; Rodriguez, R. L. and Denhardt, D. H. (1987). Vectors: A Survey of Molecular Cloning Vectors and Their Uses, Butterworth-Heinemann, Boston, Mass.; and Gilboa, E. et al. (1986). Transfer and expression of cloned genes using retro-viral vectors. Biotechniques 4(6), 504-512; and include, for example, stable or transient transfection, lipofection, electroporation, and infection with recombinant viral vectors. In addition, see U.S. Pat. Nos. 5,464,764 and 5,487,992 for positive-negative selection methods.

Typically, the nucleic acid molecule further comprises a promoter capable of driving the expression of the nucleic acid sequences wherein the promoter is a constitutively active promoter such an Elongation factor 1 (Ef1) α promoter, or a cytomegalovirus (CMV) promoter or an inducible promoter, which controls the expression of all transcription factor genes since they are organized in a polycistronic manner.

Introduction of the expression constructs of the present invention into the keratinocytes by viral infection offers several advantages over other methods such as lipofection and electroporation offering higher efficiency of transformation and propagation. According to a particular embodiment, expressing the dedifferentiating factors described herein above in the keratinocytes is performed by retroviral transduction (e.g. using a lentivirus). A contemplated cassette for infecting the keratinocytes is as set forth in SEQ ID NO: 1. Other contemplated viruses include adenoviruses and adeno-associated viruses.

According to a particular embodiment the cassette for infecting the keratinocytes comprises LOXP sites such that excision of the transgene following generation of the iPS cells may be effected by incubating with a cre-recombinase enzyme, as further described herein below. The excisable nucleic acid molecule may be any other excisable vector known in the art.

Other methods of inducing iPS cells without viral integration are also contemplated—see for example Stadtfeld et al., 2008, [Science 322, 945-949] and Okita et al., 2008, [Science 322, 949-953].

According to a specific embodiment, the keratinocytes are infected with a lentivirus comprising a polycistronic nucleic acid molecule comprising the genes encoding for the dedifferentiation factors necessary for reprogramming. Preferably a single polycistronic lentiviral vector comprising a nucleic acid molecule comprising nucleic acid sequences encoding for all transcription factors necessary for the reprogramming of the keratinocytes is used.

Preferably, the infecting. step is effected during centrifugation (e.g. at a centrifugal force of about 200-1000 g) of the culture dish containing the keratinocytes and the viruses. According to one embodiment, the centrifugation step (i.e. the length of time the keratinocytes are in contact with the virus) is not longer than two hours, and preferably not longer than one hour. Contemplated temperature of infection, according to this embodiment is between about 25° C.-37° C.

In certain embodiments, the keratinocytes are brought into contact with the lentivirus while being centrifuged under conditions comprising centrifugation for about 50 minutes at a centrifugal force of about 500 g at a temperature of about 32° C.

Immediately following the infection stage, the medium is replaced with fresh medium (devoid of virus) and fresh feeder cells. Typically, the same medium and feeder cells are used at this stage as what was originally used for culturing the fibroblasts prior to infection, although other mediums are also contemplated. Thus, for example, the infected hair follicle keratinocytes may be cultured in Green medium and 3T3 feeder cells for about 4-7 days.

The present invention contemplates more than one round of infection—for example two or three, each time removing the feeder cells prior to infection and replacing them following infection.

Following the final round of infection, the culturing conditions may be adapted for pluripotent stem cell culturing. This is advantageous, since, unlike fibroblasts, human hair follice cells cannot grow in hESC conditions. Therefore, all emerged iPS colonies are true and stable iPSCs that can be very easily observed, isolated and further expanded. Thus, for example, the medium may replaced with an embryonic stem cell medium (Thompson et al., 1998) and the 3T3 feeder cells may be replaced with mouse embryonic fibrolast (MEF) feeders. Preferably, the keratinocytes should be moved to pluripotent stem cell conditions between 3 to 6 days post infection. Culturing in pluripotent stem cell mediums may be effected for a length of time until iPS cell colonies are observed (e.g. 14-21 days).

As mentioned, the efficiency of reprogramming and/or the number of dedifferentiation factors necessary to be expressed in the keratinocytes for efficient reprogramming can be reduced by modulating for example chromatin modifications or signal transduction pathways (Feng et al., 2009). Thus, inhibitors of glycogen synthase kinase 3 (GSK-3), lysine-specific demethylase, histone methyltransferase, histone deacetylase or TGF-β; a combination of inhibitors of mitogen-activated protein kinase kinase (MAPK/ERK kinase or MEK) and GSK3; or a L-type calcium channel agonist, may be added to the embryonic stem cell medium (e.g. two days after the final infection).

According to one embodiment, the agents are added to the pluripotent stem cell medium for at least one day, at least two days, at least three days, at least four days, at least five days, at least six days, at least seven days, at least eight days, at least nine days, at least ten days, at least eleven days, at least twelve days, at least thirteen days, at least fourteen days, at least fifteen days, at least sixteen days, at least seventeen days, at least eighteen days, at least nineteen days, at least twenty days.

In particular, the histone methyltransferase is selected from the group consisting of BIX-01294, RG108 and AZA; the histone deacetylase inhibitor is selected from the group consisting of VPA, TSA and SAHA; the MEK inhibitor may be PD0325901; the TGF-β inhibitor may be A-83-01; and the L-type calcium channel agonist may be BayK8644.

It has been found in accordance with the present invention that the addition of a glycogen synthase kinase 3 (GSK-3) inhibitor and/or a lysine-specific demethylase inhibitor enables reprogramming of keratinocytes by introduction of only 3 transcription factors.

In view of the above, in certain embodiments, the number of transcription factors encoded by the nucleotide sequence is reduced to less than 4, i.e. 1, 2 or 3 transcription factors, by culturing the infected keratinocytes in the presence of small molecules such as a glycogen synthase kinase 3 (GSK-3) inhibitor and/or a lysine-specific demethylase inhibitor.

In certain embodiments, the glycogen synthase kinase 3 (GSK-3) inhibitor is CHIR99021 and the lysine-specific demethylase inhibitor is Parnate (tranylcypromine).

In certain embodiments the nucleic acid sequence encodes for 3 transcription factors selected from OCT4, SOX2 and KLF4, in particular wherein the nucleic acid sequence is transcribed from a cassette having the nucleic acid sequence as set forth in SEQ ID NO: 2.

In other embodiments, the nucleic acid sequence encodes for 2 transcription factors selected from Oct4 and Sox2.

Growth factors may also be added to the embryonic stem cells medium bFGF (e.g. Invitrogen, N.Y, USA; 8 ng/ml).

As mentioned herein above, the cassette for infecting the keratinocytes may comprise LOXP sites such that excision of the transgene following generation of the iPS cells may be effected by incubating with a cre-recombinase enzyme. Methods of excising such cassettes are provided by and Soldner et al. 2009; Brambrink et al. 2008.

In another aspect, the present invention provides induced pluripotent stem cells obtained by the method of the present invention as defined herein above. The term “induced pluripotent stem cell” as used herein refers to cells expressing pluripotent markers associated with the phenotype of hESCs, such as, but not limited to, Oct4, Sox2, Nanog, Rex1 (also known as Zinc finger protein 42 (ZFP42), TRA1-60 (Tumor Rejection Antigen 1-60), TRA1-81 (Tumor Rejection Antigen 1-81) and SSEA4 (stage-specific embryonic antigen 4), can differentiate into all 3 germ layers in vitro and in vivo and can be propagated in culture for many passages and keep normal karyotype. As shown herein below in Example 4, four selected HFKT-iPSCs clones were fully characterized for their expression of keratinocyte and pluripotency markers, relative to the source HFKTs as well as to pluripotent hESCs. The present inventors found that keratinocyte markers such as K14 and P63 were still highly expressed in HFKT-iPSCs relative to hESCs, suggesting retention of an “epigenetic memory”, as previously described (Hochedlinger et al. 2009; Marchetto et al. 2009). However, all pluripotent markers analyzed were positively and similarly expressed in hESCs and HFKT-iPSCs, indicating that true pluripotent iPSCs clones were generated. In particular, the iPSCs clones were found to express typical hESC markers, such as Oct4, Sox2, Nanog, Rex1, TRA1-60 and TRA1-81.

Microarray analysis may be performed on the iPSCs of this aspect of the present invention in order to determine which genes are specifically expressed in the cells. Using this method, iPSCs generated from human hair follicle keratinocytes may be compared with other plupripotent stem cells, embryonic stem cells (ESCs) and iPSCs generated from skin. Genes that are upregulated or downregulated by more than 2 fold or more may be considered to be significantly changed. Confirmation of the results may be effected using any method known in the art and include for example RT-PCR analysis and immunostaining.

Thus, in certain embodiments the induced pluripotent stem cells of the present invention are characterized by the expression of typical hESC markers, such as Oct4, Sox2, Nanog, Rex1, TRA1-60 and TRA1-81.

Since a lentiviral vector with a constitutively active promoter was used for reprogramming, it was predicted that the transgenes expression levels in the HFKT-iPSCs will be higher relative to their hESCs counterparts. Interestingly, excluding high Klf4 expression in only one clone (KTN7), transcript levels of the reprogramming factors in all investigated clones were similar to those found in hESCs. The results presented hereinafter in the Examples indicate that silencing of the exogenous transgenes indeed occurs in the HFKT-iPSCs clones generated by the human STEMCCA vector. The exogenous transgenes were further silenced during passaging of the iPSCs. These data demonstrate that, human iPSCs adopt a gene expression profile that with the time of culture becomes more similar to that of hESCs.

The HFKT-iPSCs of the present invention could further differentiate spontaneously into all 3 germ layers in vitro and in vivo. The differentiation capacity of the HFKT-iPSC clones was further demonstrated herein in Example 5 by their ability to specifically differentiate into cardiomyocytes (CMs), which were characterized for their molecular and functional properties. The positive immunostaining of multiple myofilament proteins suggested that a preliminary organization of a sarcomeric structure can develop in HFKT-iPSC-CMs, similarly to hESC-derived CMs and iPSC derived-CMs from other cell sources (Germanguz et al. 2009; Zhang et al. 2009). Evidence for the functionality of the HFKT-iPSC-CMs was provided by the robust extracellular electrograms recorded by means of the multiple-electrode array (MEA) data acquisition system and by the action potentials recorded by whole cell current clamp. Moreover, the excitation-contraction coupling, typical of CMs, as well as the responsiveness to β-adrenergic stimulation were illustrated herein by measuring the cells' [Ca²⁺]ᵢ transients and contractions. Thus, the present inventors demonstrate for the first time the ability of HFKT-iPSCs to differentiate into functional CMs and to serve as an alternative source of cells for therapeuthic and research purposes.

In certain embodiments, the induced pluripotent stem cells of the present invention are capable of differentiating into embryoid bodies or teratomas, and the embryoid bodies and teratomas comprise derivatives of all three germ layers. Embryoid bodies are aggregates of stem cells that differentiate into different cell types and to a limited extent recapitulate embryonic development. A teratoma is a tumor consisting of different types of tissue, as of skin, hair, and muscle, caused by the development of independent germ cells.

The phrase “derivatives of all three germ layers” as used herein refers to differentiating or differentiated cells derived, i.e. developed, from any of the three germ layers, i.e. endoderm, mesoderm and ectoderm.

It has been found in accordance with the present invention that the induced pluripotent stem cells are capable of differentiating into functional cardiomyocytes, neuronal tissue, endodermal epithelium, adipose or muscle tissues and other lineage specific cells. It will be appreciated that the present invention contemplates the use of any ex vivo differentiation protocol known in the art for the generation of such lineage specific cells.

In certain embodiments, the embryoid bodies derived from the induced pluripotent stem cells of the present invention are capable of differentiating into functional cardiomyocytes, neuronal tissue, endodermal epithelium, adipose tissue, muscle tissue including skeletal, smooth and cardiac muscle, endothelial progenitor cells, mesenchymal progenitor cells bone, cartilage, tendon and ligament tissues and particularly extracellular matrix producing cells.

The method of the present invention may therefore, in view of the above, provide stem cells that may be used for tissue regeneration, gene therapy, cell therapy, drug screening and disease modeling. For example, the cells can be used for generation of organs and tissues for transplantation, and thus provides a promising alternative therapy for diabetes, neurodegenerative diseases like Parkinson's disease, liver disease, heart disease, orthopedic diseases and autoimmune disorders, to name a few. Alternatively, the cells may be used to provide functional genes to a tissue in need for gene-replacement therapy.

In one aspect, the present invention provides a pharmaceutical composition comprising a pharmaceutically acceptable carrier and the induced pluripotent stem cells generated according to the method as defined herein.

As stated above, the induced pluripotent stem cells of the present invention may be used to regenerate any damaged tissue. In particular, the present invention is directed to a method for generating functional cardiomyocytes comprising inducing pluripotent stem cells obtained according the methods defined herein to differentiate into functional cardiomyocytes, thereby obtaining functional cardiomyocytes; and to methods for repairing damaged cardiac tissue comprising replacing the damaged tissue with functional cardiomyocytes obtained according to the method of the present invention, wherein the functional cardiomyocytes forms a functional cardiac tissue, thereby repairing the damaged cardiac tissue.

For example, the method can be used to repopulate heart muscle cells by either direct injection into the area of tissue damage or by systemic injection, allowing the cells to home to the cardiac tissues.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Derivation of Keratinocytes from Plucked Hair Follicles.

Human plucked hairs were acquired from healthy volunteers who signed consent forms according to approval 3611 by the Helsinki Committee for Experiments on human subjects at Rambam Health Care Campus, Haifa, Israel. Hair follicle keratinocytes (HFKTs) were derived as described (Limat et al. 1986) with minor modifications. Ten hairs with visible outer root sheath were plucked from the scalp. The bulk of the hair was cut off and the follicles were immersed in a 10 cm Petri dish with DMEM medium containing 25 mM HEPES, 1 mM L-Glutamine and 400 U/ml penicillin, 400 μg/ml streptomycin (PS) for 4-18 hours in 37° C. The follicles were washed with PBS, then covered with 0.1% Trypsin and 0.02% EDTA (diluted with PBS) and incubated for 30 minutes at 37° C. A single cell suspension culture was obtained by vigorously pipetting the follicles with DMEM supplemented with 10% FBS. The dissociated keratinocytes were centrifuged for 10 min at 200 g and seeded in 3 wells of a 6-well plate on an inactivated 3T3 feeder layer (2*10⁴ 3T3 cells/cm²) with Green medium (60% DMEM, 30% DMEM F-12, 10% Fetal Bovine Serum, 1 mM Sodium Pyruvate, 2 mM L-Glutamine, 5 μg insulin, 0.5 μg/ml Hydrocortisone, 0.2 nM Adenine, 2 nM triiodothyronine (T3), 10 ng/ml Epidermal Growth Factor and 100 U/ml penicillin, 100 μg/ml streptomycin). For splitting, 3T3 cells were removed after incubation with 0.02% EDTA for 5 minutes in 37° C. The culture was washed with PBS, after which the detached keratinocytes were dissociated into single cells by incubation with 0.1% Trypsin and 0.02% EDTA in PBS at 37° C. for 10-15 minutes.

Cells:

Human foreskin fibroblast (HFF) cells were obtained from ATCC (PCS-201-010). 293T cells (CRL-11268). H9.2 hESCs were used (Amit et al. 2000; Amit et al. 2002). Fibroblast cells (HDF) and HFKTs were obtained from two healthy individuals, from either skin punched biopsy or plucked hair, respectively.

Vectors:

for HFF infection: Zeomycin resistance-PBabe-eco (AddGene ID: 10687) and PMX OCT4/Sox2/KLF4/c-Myc (Addgene ID 17217, 17218, 17219, 17220,), For HFKT and HDF infection: 1) PMX OCT4/Sox2/KLF4/c-Myc together with pUMVC and pCMV-VSVG, (AddGene no. 8449 and 8454). 2) pMSCV/Oct4/Sox2/KLF4/c-Myc (murine stem cell virus, Addgene ID: 20072, 20073, 20074, 20076) 3) human single polycistronic lentiviral vector harboring the STEMCCA cassette (SEQ ID NO: 1). Lentivirus was produced using a five plasmid transfection system in 293T packaging cells as previously described (Mostoslaysky et al. 2006).

Generation of iPSCs from Human Foreskin Fibroblast (HFF) Cells

HFF cells were infected with the pBabe-Eco plasmid, followed by 7 days selection with 400 ng/ml Zoecin (Invitrogen). For iPSC generation, phoenix Eco cells were transfected with one of the four retroviral vectors: pMX-Oct4/Sox2/Klf4/c-Myc. Two days post transfection, 100,000 HFF-eco cells were infected as previously described (Takahashi et al. 2007). Two days following the infection, the HFF-eco cells were split at various dilutions and cultured on either a MEF feeder layer or fibronectin-coated plates. Five days post infection, the medium was replaced with either hESC medium or MEF-conditioned medium. Approximately 14 days post infection, small ESC-resembling colonies emerged which later were mechanically isolated and cultured on MEF in hESC conditions for further analysis.

A reprogramming efficiency assay was carried out using live staining of Tral-60, as described elsewhere (Lowry et al. 2008).

Generation of iPSCs from Human Dermal Fibroblast (HDF) Cells.

HDF cells were derived as previously described (Park et al. 2008). Cells were infected with either the lentiviral vector harboring the STEMCCA cassette which contains the four factors Oct4, Sox2, Klf4 and c-Myc, or with four separate retrovirus pMXs vectors expressing Yamanaka's reprogramming gene set (Oct4, Sox2, Klf4, c-Myc). For iPSC generation, the STEMCCA vector was transfected into 293T cells as described above for iPSCs derived from HFKTs. The pMXs vectors were transfected into Phoenix-Ampho cells and 70,000 HDF cells were infected as described above for HFF cells.

Generation of iPSCs from HFKTs

On the first day 30,000 HFKTs were seeded on an inactivated 3T3 feeder layer (20,000 cells/cm²) supplemented with Green medium, in one well of a 6-well plate (Limat and Noser 1986). On the second day, viruses were produced as follows: The humanized version of a single lentiviral vector STEMCCA Cassette (SEQ ID NO: 1) was generated following the transfection of 293T cells with five plasmids: STEM-CCA: Gag-Pol: REV: TAT: VSVG, at ratios of 20:1:1:1:2, respectively (Mostoslaysky et al. 2006). The total plasmid amount was 15 μg DNA. Transfection was done by a jetPEI™ Reagent (Polyplus Transfection™, France). The pMSCV retroviruses were generated in Pheonix-Ampho cells that were transfected with the vectors using a jetPEI™ Reagent. Medium supernatants containing viruses from all four transgenes were collected and mixed at a ratio of 1:1:1:1 (pMSCV/Oct4/Sox2/Klf4/c-Myc). The retroviral vectors pMX-Oct4/Sox2/Klf4/c-Myc were generated as previously described (Park et al. 2008). The medium was replaced with a fresh one 24 hrs poist-transfection (day 3). At 48 hrs post-transfection (day 4), the accumulated viral particles were filtrated through a 0.45 μm filter, supplemented with 2 μg/ml polybrene and used for infection of HFKTs. Immediately before infection 3T3 feeder cells were removed using 0.02% EDTA. The infection was performed during centrifugation for 50 min with 500 g, at 32° C. Thereafter, the medium was replaced with fresh Green medium, and fresh inactivated 3T3 feeder cells were added. The infection was repeated on the following day (day 5). On day 5 post-infection (day 8), infected keratinocytes were detached by 0.1% Trypsin and 0.02% EDTA in PBS (Biological Industries, Beit Haemek, Israel), at 37° C. for 10 min, centrifuged for 10 min at 200 g, and seeded on an inactivated mouse embryonic fibroblast (MEF) feeder with Green medium, in six wells of a 6-well plate. On the following day the medium was replaced with hESC medium (Thomson et al. 1998) containing 8 ng/ml bFGF (Invitrogen, N.Y, USA). The medium was replaced every second day. Finally, 21-25 days after seeding the infected keratinocytes, hESC-like colonies emerged and could be further expanded and analyzed.

Differentiation of iPSCs.

Differentiation of iPSCs into EBs was carried out as previously described (Itskovitz-Eldor et al. 2000). Briefly, human iPSCs were detached by 0.2% type IV collagenase (Worthington Biochemical, Lakewood, N.J., USA) and suspended in order to allow their aggregation. The resultant EBs were grown in 80% DMEM (Gibco-BRL, Grand Island, N.Y., USA), 20% FBS (Hyclone, Cramlington, UK), 1 mmol/l L-glutamine and 1% non-essential amino acid (both from Gibco-BRL, Grand Island, N.Y., USA). For spontaneous differentiation, the EBs were cultured in suspension for 14 days and then dissociated using 1 mg/ml collagenase B (Roche, Mannheim, Germany) in PBS supplemented with DNase for 10 min at 37° C. The dissociated EBs were cultured on 0.1% gelatin-coated (Sigma-Aldrich St. Louis, Mo., USA) coverslips for an additional seven days, and then immunostaining assays were performed. For cardiac differentiation, the EBs were cultured in suspension for seven days and subsequently plated on 0.1% gelatin-coated plates, during which spontaneously contracting EBs were observed. In order to assess the efficacy of the cardiac differentiation, the plated EBs were monitored microscopically and, after 2-3 weeks of culturing, the number of contracting EBs was counted out of the total number of plated EBs. For teratoma generation, 2×10⁶ iPSCs were injected into the flanks of recipient SCID mice. Tumors were isolated for histological analysis 6-8 weeks later, fixed in 4% paraformaldehyde, embedded in paraffin and sectioned. Paraffin sections were deparaffinized by Xylol/Xylene, then rehydrated with propanol and washed with distilled water. The sections were stained with haematoxylin and eosin according to standard protocols.

RNA Analysis.

RNA was isolated using Aurum™ Total RNA Mini Kit (BIO-RAD, Hercules, Calif., USA) and reverse transcribed by the iScript™ cDNA synthesis kit (BIO-RAD,), according to the manufacturer's instructions. PCR was performed by DreamTaq™ Green Master Mix (Fermentas, Ontario, Canada). Quantitative Real Time (QRT) PCR analysis was performed in triplicate and normalized by the internal endogenous GAPDH gene expression. The reaction was performed in an ABI Prism 7000 (Applied Biosystems, Warrington, UK) with Power SYBR® Green Master Mix (Applied Biosystems). Analysis was conducted using the Relative Quantification (RQ) study in the Sequence Detection Software (V. 1.2; Applied Biosystems). The primers used for RNA analysis are listed in Table 1.

TABLE 1
Primers used for RNA analysis

| Gene | 5′ primer | SIN¹ | 3′ primer | SIN¹ |
|---|---|---|---|---|
| GAPDH | CCACATCGC | 3 | GGCAACAATA | 4 |
| | TCAGAACCAT | | TCCATTTACCAG | |
| Oct4 | CTCACCCTGG | 5 | CTCCAGGTTGC | 6 |
| | GGGTTCTAT | | CTCTCTCACT | |
| Nanog | TGAGTGTGG | 7 | TGAATAAG | 8 |
| | ATCCA | | CAGATC | |
| Klf4 | CTCAAGGCA | 9 | AGTGCCTG | 10 |
| | CACCTG | | GTCAGTT | |
| c-myc | ACTCTGAGGA | 11 | TGGAGACGT | 12 |
| | GGAACAAG | | GGCACCTCTT | |
| Sox2 | GGGAGGGGT | 13 | CACAGCAA | 14 |
| | GCAAA | | ATGACAG | |
| Rex1 | ACAGTCCAG | 15 | CTTGTCTT | 16 |
| | CAGGT | | TGCCCGT | |
| K14 | GACCATTGAG | 17 | CATACTTGG | 18 |
| | GACCTGAGGA | | TGCGGAAGTCA | |
| p63 | TTTCCCACC | 19 | TGCGGCGAG | 20 |
| | CCGAGATGA | | CATCCAT | |

¹SIN, SEQ ID NO:

Protein Analysis.

Immunofluorescence:

Cells were fixed with 4% paraformaldehyde for 15 min and permeabilized in 1% triton X-100 (Sigma-Aldrich, St. Louis, Mo., USA), then diluted in PBS for 10 min. Primary antibodies were diluted in PBS with 1% Triton and incubated overnight at 4° C. or for one hr at room temperature. The antibodies used are listed in Table 2.

Donkey anti Rabbit Cye 3 (Chemicon International) and Donkey anti Mouse/Goat Alexa fluor 488 (Invitrogen, Carlsbad, Calif., USA) conjugated antibodies were used as secondary antibodies (1:100). Cells were also stained with DAPI (1:1000) (Boehringer, Mannheim, Germany) for nuclei staining and examined with Zeiss Axiovert 200 fluorescent microscope or with Zeiss LSM 510 Meta laser scanning confocal system (Carl Zeiss, Munich, Germany).

TABLE 2
Antibodies used for immunostaining

| Antibody | Company | Dilution |
|---|---|---|
| Rabbit anti Oct3/4 | Santa Cruz | 1:100 |
| Goat anti Nanog | R&D | 1:20 |
| Mouse anti Sox2 | Millipore | 1:100 |
| Mouse anti tumor recognition antigen | Millipore | 1:100 |
| (TRA) 1 | | |
| Mouse anti tumor recognition antigen | Millipore | 1:100 |
| (TRA) 1 | | |
| Mouse anti Cytokeratin 14 | chemicon | 1:100 |
| Rabbit anti KLF4 | chemicon | 1:100 |
| Mouse anti C-Myc | Chemicon | 1:100 |
| Mouse anti Nestin | Chemicon | 1:100 |
| Rabbit anti Tubulin IIIβ | Covance | 1:2000 |
| Mouse anti CD31 | Dako | 1:100 |
| Mouse anti Smooth muscle actin (SMA) | Dako | 1:100 |
| Rabbit anti Alpha-fetoprotein (AFP) | Dako | 1:1 |
| Mouse anti Glucagone | Dako | 1:50 |
| rabbit anti-cardiac troponin I | Abcam | 1:400 |
| mouse anti sarcomeric α-actinin | Sigma-Aldrich | 1:600 |
| mouse anti-α/β myosin heavy chain | Chemicon | 1:40 |

Fluorescence-Activated Cell Sorting (FACS) Analysis:

The iPSCs and hESCs were detached using 0.2% IV collagenase and dissociated into single cells using 0.25% Trypsin and 0.05% RDTA. The HFKTs were detached using 0.1% Trypsin and 0.02% EDTA in PBS (following removal of 3T3 feeder cells with 0.02% EDTA). The cells were fixed using 4% paraformaldehyde for 15 min. They were monitored by flow cytofluorometry on a FACScan system using CellQuest software (BD Biosciences, San Jose, Calif., USA). Antibodies used for FACS are listed in Table 3.

Measurements of [Ca⁺²]i Transients and Contractions.

[Ca²⁺]ᵢ transients and contractions were measured in small dissociated contracting areas of EBs by means of fura-2 fluorescence (Biotium, Hayward, Calif., USA) and a video edge detector, respectively, as previously described (Dolnikov et al. 2006; Sedan et al. 2008).

Microelectrode Array (MEA) Recordings.

Unipolar electrograms were recorded from HFKT-iPSC-derived cardiomyocytes (HFKT-iPSC-CMs) plated on MicroElectrode Arrays (MEAs) (Multi Channel Systems, Reutlingen, Germany), as previously described (Meiry et al. 2001; Reisner et al. 2009).

TABLE 3
Antibodies for FACS analysis

| Antibody | Company | Dilution |
|---|---|---|
| Mouse anti CD90 PE conjugated | Biolegend | 1:100 |
| Mouse anti CD29 PE conjugated | eBioscience | 1:100 |
| Mouse anti stage-specific | Hybridoma Bank, | 1:100 |
| embryonic antigen 4 (SSEA4) | Iowa City | |
| *Mouse anti Cytokeratin 14 (K-14) | Chemicon | 1:100 |

*For cellular staining of K14, the cells were permeabilized in PBS with 1% saponin (Sigma-Aldrich) and 0.5% BSA for 10 min. Cells were stained with anti K-14 diluted in PBS with 1% saponin and 0.5% BSA (Both from Sigma-Aldrich).
Donkey anti Mouse Alexa fluor 488 (Invitrogen) conjugated antibody was used as secondary antibody (1:100) diluted in PBS with 0.5% BSA, or with 1% saponin for K-14 staining.

Whole-Cell Current Clamp Recordings.

For the current clamp studies, spontaneously beating small cell clusters or isolated cells produced after dissociation of the HFKT-iPSC-CMs were studied following plating on top of fibronectin-coated glass coverslips. The patch pipette solution consisted of (mM): 120 KCl, 1 MgCl₂, 3 Mg-ATP, 10 HEPES, 10 EGTA (pH=7.3). The bath solution consisted of (mM): 140 NaCl, 5.4 KCl, 1.8 CaCl₂, 1 MgCl₂, 10 HEPES, 10 glucose (pH=7.4) (All materials from Sigma-Aldrich, St. Louis, Mo., USA). Action potentials were recorded using the current clamp mode. Axopatch 200B, Digidata1322, and pClamp 10 (Molecular devices, Sunnyvale, Calif., USA) were used for data amplification, acquisition and analysis.

Short Tandem Repeat Analysis and Karyotyping.

Short tandem repeat analysis (STR) was performed using the sequences obtained from different chromosomes and analyzed by an ABI PRISM 3130 genetic analyzer, according to the manufacturer's instructions (Applied Biosystems, Warrington, UK). The STR primers are listed in Table 4. Karyotyping was performed as previously described (Amit et al. 2003).

TABLE 4
Sequences for short tandem repeat (STR) analysis

| | 5′ primers | SIN¹ | 3′ primers | SIN |
|---|---|---|---|---|
| D1S2692 | GCTAACAAAAACCCACATCT | 21 | GCTAACAAAAACCCACATCT | 22 |
| D1S2828 | GGCTCCTGAACCTGGG | 23 | AGCTTTGGCTGACCTTCC | 24 |
| D3S1613 | TGTGATAAGGACCAAGGC | 25 | GAGCAAATTGCAGAATGAG | 26 |
| D5S346 | ACTCACTCTAGTGATAAATCGGG | 27 | GGAACCAGAAACTGTGGCAT | 28 |
| D6S426 | CATGTGCTCTGCACCATAAG | 29 | GGAACCAGAAACTGTGGCAT | 30 |
| D7S486 | AATCTGTTCTGGCAATGG | 31 | TTATGTTTACTTTCTCAGTGGG | 32 |
| D11S988 | CAGAAAATAGTTCAGACCACCA | 33 | GGGACAAGAGAAAGTTGAACA | 34 |
| D11S2362 | TGGACTATAGGACCCCCTTC | 35 | GAGAACAGCCTGTCACACCT | 36 |
| D15S211 | AAGCAGGTGGAATCCTTG | 37 | AAAAGCCCCAGGTAGGG | 38 |
| D15S1023 | GGTATTGTTTTGGACCACATCTTAG | 39 | GGGAGGCTGAGACAGTTTC | 40 |
| D19s865 | GCTATTTGGGGTCTCTATCAATG | 41 | GAAATCGCACAGTATTTGTCTCAC | 42 |
| DXS1193 | AATTCTGACTCTGGGGC | 43 | TTATTTTAAGGTGAGTATGGTGTGT | 44 |

¹SIN, SEQ ID NO:

### Example 1

### The Derivation of Keratinocytes from the ORS of Plucked Hair Follicle and their Characterization

In order to generate iPSCs from hair, the present inventors followed Aasen et al.'s protocol (Aasen et al. 2008) for generating iPSCs from plucked hair follicles. Plucked hair was cultured on Matrigel-coated dishes supplemented with MEF-conditioned media for at least five days until cells proliferated out of the outer root sheath (ORS), but an insufficient number of viable and proliferative cells could be isolated. The present inventors obtained a single cell suspension of keratinocytes by plucking and selecting at least 10 single hairs with a visible bulb and intact ORS (FIG. 1A), incubating them with DMEM-supplemented with penicillin, streptomycin, HEPES and L-Glu for 24 hr, and then removing the cells from the ORS enzymatically with trypsin-EDTA (FIG. 1B). These isolated keratinocytes were seeded on inactivated 3T3 feeder cells (FIGS. 1C, D) and could be further cultured up to four passages (Limat and Noser 1986),

In order to verify their identity, the hair follicle-derived cells were analyzed for the keratinocyte markers K14 and P63. Analysis was performed by QRT-PCR relative to HaCat cells (a human keratinocyte cell line) and hESCs. The results revealed high expression levels of K14 and P63 transcripts, similar to their expression in HaCat keratinocytes. In contrast, both transcripts were barely expressed in the hESCs (FIG. 1E). Next, the expression levels of the reprogramming factors were tested. The expression of Klf4 was 10 fold higher in the HFKT and HaCat cells than in the hESCs, whereas c-Myc was similarly expressed in all three cell types. Oct4 was not expressed in HFKT, and Sox2 was slightly expressed relative to the hESCs (FIG. 1F).

### Example 2

### Optimization of the Reprogramming Procedure

With the purpose of developing an efficient HFKT reprogramming protocol, the present inventors used various viral vectors and growth conditions to optimize the reprogramming of common target cells, such as human foreskin fibroblasts (FIFE) and human dermal fibroblasts (HDF). The viral vectors analyzed were the pMX retroviral vectors harboring four (Klf4, Oct3/4, Sox2 and c-Myc) or three (Klf4, Oct3/4 and Sox2) reprogramming factors (Takahashi et al. 2007; Nakagawa et al. 2008), The pMSCV retroviral vector set modified by Aasen et al. (Aasen et al. 2008) and the humanized version of a single lentiviral STEMCCA vector (SEQ ID NO: 1).

TABLE 5
Calibration of post-infection growth-conditions

| | Cells | | | Cell | |
|---|---|---|---|---|---|
| | maintenance | | | subculturing | Reprogramming |
| Cell | (Fresh/thaw | | Growth | ratio | Efficiency |
| source | after freezing) | Viral vector | conditions | (post infection) | % + SD |
| HFF | Thaw cells | pMX 4F | MEF feeder | 1:6 | 0.04% ± 0.03 |
| | Fresh cells were | (OSKM) | layer | | |
| | not determined | | Fibronectin + | | 1% ± 0.46 |
| | (ND) | | MEF CM | | |
| | | pMX 3F | MEF feeder | | 0.022% ± 0.0125 |
| | | (OSK) | layer | | |
| | | | Fibronectin + | | 0.19% ± 0.01 |
| | | | MEF CM | | |
| HDF | Fresh cells | STEMCCA | MEF feeder | 1:6 | 3.22% ± 0.62 |
| | | | layer | 1:36 | 1.88% ± 0.43 |
| | | | Fibronectin + | 1:6 | ND* |
| | | | MEF CM | 1:36 | 1.13% ± 0.44 |
| | Thaw cells | | MEF feeder | 1:6 | 0.08% ± 0.01 |
| | | | layer | 1:36 | 0.3% ± 0.08 |
| | | | Fibronectin + | 1:6 | ND* |
| | | | MEF CM | 1:36 | 0.017% ± 0.005 |
| | Fresh and | pMX 4F | MEF feeder | 1:6 and 1:36 | No iPSCs |
| | Thaw cells | (OSKM) | Layer or | | |
| | | pMSCV 4F | Fibronectin + | | |
| | | (OSKM) | MEF CM | | |
| HFKT | Fresh cells ** | STEMCCA | MEF feeder | 1:4-1:6 | 0.03% ± 0.002 |
| | | | layer | | |
| | | | Fibronectin + | | 0.03% ± 0.001 |
| | | | MEF CM | | |
| | | pMX 4F | MEF feeder | 1:6 | No iPSCs |
| | | (OSKM) | layer | | |
| | | pMSCV 4F | Or: | | |
| | | (OSKM) | Fibronectin + | | |
| | | | MEF CM | | |

*Efficiency was not determined (ND) because of massive overgrowth.
** Only fresh HFKTs revealed the iPSC colonies

The HFF cells were most efficiently reprogrammed following the establishment of an HFF-Ecotropic receptor (HFF-Eco) stable line. For reprogramming, these cells were further infected with pMX retroviral vectors harboring four or three reprogramming factors. Following infection, various growth conditions were tested (Table 5). It was found that plating the cells post-infection at low density was crucial in order to avoid overgrowth of non-iPSCs which frequently cover the true iPSC colonies. Moreover, reprogramming efficiency was increased by ˜10-50-fold by culturing the cells in feeder-free conditions, as compared to cells cultured on MEF feeder layers supplemented with hESC medium. Although HFF reprogramming efficiency declined when only three factors were introduced, as previously described (Nakagawa et al, 2008; Soldner et al. 2009), it increased by 10-fold when the cells were cultured in feeder-free conditions rather than on MEF feeder layers (Table 5—HFF).

The three different viral vectors were tested for the reprogramming of HDF and HFKTs, using an appropriate packaging vector as previously described (Huangfu et al. 2008b). It was found that both cell types, HDF and HFKT, were efficiently reprogrammed with the STEMCCA vector, whereas no iPSC colonies were generated using either pMX or pMSCV vector sets (Table 5). Further, it was found that fresh cells, infected soon after isolation, were more efficiently reprogrammed than frozen ones. Incubating the HDF and HFKTs in feeder-free conditions did not improve reprogramming efficiency, as was found for the HFF cells, thus indicating that efficient reprogramming protocols should be exclusively optimized for each cell type.

Cell density post-infection was critical only for the fibroblast cells, as overly crowded cultures led to the overgrowth of fast growing non-iPSCs that covered the true iPSC colonies. However, since HFKTs are very sensitive cells that do not propagate in hESC conditions, no growth of non-iPScs occurred in the reprogramming culture. Rather, only true iPSCs emerged following reprogramming with the STEMCCA vector. This observation points to a significant advantage of the HFKTs as a cell source for reprogramming.

The following conditions were found to improve the efficiency of HFKTs reprogramming into iPSCs (see Table 6):

1. Culturing the HFKTs following infection for 4-7 days in green medium before transferring them to MEF feeder layer and hESCs conditions. Culturing the HFKTs on MEF+hESC conditions inhibit dramatically their growth. It was found that growing the infected cells for several days at their optimal medium (green medium) promote their growth post infection. Although their transfer to hESCs growth conditions after 4-7 days inhibit their growth immediately, more iPSCs were generated at these conditions.

2. The addition of 8-10 μM CHIR99021 (StemGent), which is a specific glycogen synthase kinase 3 (GSK-3) inhibitor

3. The addition of CHIR99021 in combination with Parnate (Sigma; also named tranylcypromine), which is an inhibitor of lysine-specific demethylase.

4. Following the addition of Chir99021 alone or Chir+ parnate combination the present inventors were able to generate iPSCs with no c-Myc, and also, at a very low efficiency, to generate iPSCs with no myc and no Klf4 (2 factors iPSCs). The infection was done by a similar polycistronic vector including only 3 reprogramming factors: Oct4, Klf4 and Sox2, without c-Myc (the cherry vector), or by a similar polycistronic vector including only 2 reprogramming factors: Oct4 and Sox2, respectively.

Chir99021 was shown to increase reprogramming efficiency of mouse embryonic able the reprogramming of human primary skin derived keratinocytes only by 2 factors—Klf4 and Oct4 (Li W. et al, Stem cells 2009 27:2992-3000).

TABLE 6
Conditions found to improve the efficiency
of HFKTs reprogramming into iPSCs

| Validated | | |
|---|---|---|
| iPSCs colonies | Treatment | Cells |
| 2 | CHIR | KTN + Stemcca |
| 16 | CHIR + Parnate | (4 reprogramming |
| 6 | Green medium for | factors) |
| | a week, post | |
| | infection | |
| 0 | No treatment | |
| 6 | CHIR | KTN + Cherry |
| 8 | CHIR + Parnate | (3 reprogramming |
| | | factors, no c-Myc) |
| 0 | No treatment | |

Note that this experiment suffer from low reprogramming efficiency because we used frozen virus and also reduced virus concentration. Therefore we couldn't get any iPSCs with normal treatement. However, we could generate iPSCs when adding the small molecules or culturing the cells with green medium for a week post infection and then transfer them to hESCs conditions.

### Example 3

### Induction of Pluripotent Stem Cells from HFKTs, and their Identity Relative to HFKT Parental Cells and hESCs

The procedure for HFKT reprogramming is illustrated in FIG. 2A. KTN and KTR keratinocytes were derived from plucked hairs of two healthy women, aged 36 and 41, designated “N” and “R”, respectively. Starting with 30,000 keratinocytes which were infected with the STEMCCA vector, ˜5-9 iPSC colonies were isolated in five independent experiments. Importantly, all the colonies that emerged following ˜30 days of incubation were true iPSCs exhibiting morphological features resembling those of hESCs (FIG. 2B). All the iPSC colonies were picked mechanically and were transferred to the MEF feeder-layer for further expansion and analysis. Their karyotypes were analyzed and were found to be normal excluding two clones, one with a lost X chromosome (45, X0) and the other with an unstable karyotype (data not shown).

Specific characterization of the HFKT-iPSCs, as compared with their parental KTR/KTN cells was performed by analysis of the K14 protein. K14 staining showed strong cytoplasmic expression in the HFKTs, which was reduced in the HFKT-iPSCs (FIG. 2C). FACS analysis revealed that while 77% of the HFKTs expressed K14 protein, only 50% of their iPSC derivatives were K14 positive. The level of K14 protein in the hESCs was 30% (FIG. 2D). Klf4 and c-Myc proteins were also analyzed by immunostaining and found to be highly expressed in the HFKT donor cells, and their expression levels were mostly sustained following reprogramming and in the hESCs (FIG. 2C). Next other ESC markers were analyzed by FACS analysis. It was found that only 25% of HFKT donor cells expressed CD90 whereas SSEA4 protein was not expressed in these cells, the HFKT-iPSCs positively expressed CD90 (91%) and SSEA4 (73%). These markers were similarly expressed in the hESCs, with 83% for CD90 and 68% for SSEA4. CD29 expression was reduced following pluripotency, whereas the HFKTs were 95% positive for this marker, their derived iPSCs and hESCs showed a notably reduced CD29 signal. Although 70% of the cells were positive, the signal intensity was ˜7 fold lower (Geome mean parameter was 34.3 for the HFKTs and only 5 for the iPSCs and hESCs). These results are in agreement with data obtained for human foreskin keratinocytes and their iPSC derivatives (Aasen et al. 2008). DNA fingerprint analysis (Short tandem repeat—STR) confirmed that the HFKT-iPSC clones were derived from two different sources, KTN and KTR cells, and that the genetic profile of the iPSC clones was identical to their donor cells (Table 7).

TABLE 7
STR analysis for iPSC clones and their original source cells.

| | | | KTR Source | | | KTN Source | Non relevant |
|---|---|---|---|---|---|---|---|
| | KTR-13 | KTR-12 | cells | KTN-3 | KTN-7 | cells | DNA |
| D1S2692 | 191, 193 | 191, 193 | 191, 193 | 195, 197 | 195, 197 | 195, 197 | 195, 205 |
| D1S2828 | 243, 247 | 243, 247 | 243, 247 | 247, 261 | 247, 261 | 247, 261 | 247, 249 |
| D3S1613 | 233, 239 | 233, 239 | 233, 239 | 239, 239 | 239, 239 | 239, 239 | 237, 239 |
| D5S346 | 108, 118 | 108, 118 | 108, 118 | 112, 112 | 112, 112 | 112, 112 | 108, 108 |
| D6S426 | 200, 200 | 200, 200 | 200, 200 | 205, 209 | 205, 209 | 205, 209 | 207, 209 |
| D7S486 | 137, 139 | 137, 139 | 137, 139 | 129, 143 | 129, 143 | 129, 143 | 129, 137 |
| D11S988 | 109, 115 | 109, 115 | 109, 115 | 109, 124 | 109, 124 | 109, 124 | 124, 124 |
| D11S2362 | 220, 226 | 220, 226 | 220, 226 | 214, 223 | 214, 223 | 214, 223 | 214, 215 |
| D15S211 | 227, 233 | 227, 233 | 227, 233 | 235, 241 | 235, 241 | 235, 241 | 229, 245 |
| D15S1023 | 276, 285 | 276, 285 | 276, 285 | 279, 281 | 279, 281 | 279, 281 | 276, 281 |
| D19S865 | 221, 221 | 221, 221 | 221, 221 | 221, 225 | 221, 225 | 221, 225 | 225, 227 |
| DXS1193 | 123, 123 | 123, 123 | 123, 123 | 121, 123 | 121, 123 | 121, 123 | 123, 123 |

STR analysis was performed on iPSC clones KTR12, KTR13 and their HFKT source cells KTR, as well as on iPSC clones KTN3, KTN7 and their HFKT source cells KTN. As negative control non relevant DNA generated for non related individual, was used.

### Example 4

### Pluripotency of HFKT-iPSC Clones

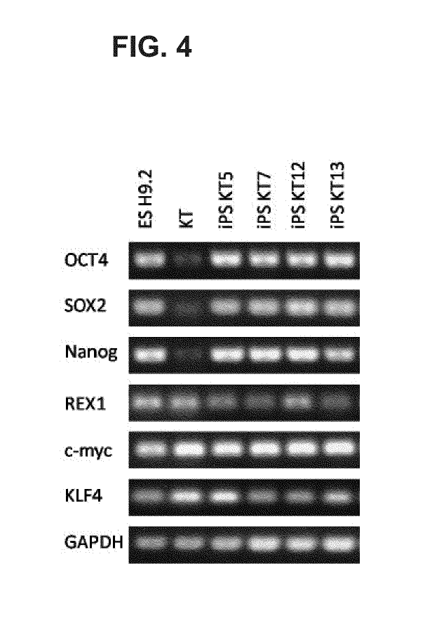

In order to obtain a detailed characterization, four HFKT-iPSC clones were selected: KTN3 and KTN7 derived from a KTN cell source, and KTR12 and KTR13 derived from a KTR cell source. These clones were maintained for up to 40 passages and resembled the hESCs in morphology and karyotypic stability. The iPSC clones were found to express typical hESC markers, such as Oct4, Sox2, Nanog, Rex1, TRA1-60 and TRA1-81, as demonstrated by immunostaining (FIG. 3A) and RT-PCR analysis (FIG. 4). The transcript levels of the reprogramming factors of all four HFKT-iPSC clones were analyzed by QRT-PCR, at three different passages, namely, 5-7, 12-17 and 22-25, and they were compared with their hESC counterparts (FIG. 3B). The results showed that the transcript levels of Oct4 and Sox2 were similar to those of the hESCs. Except for reduced levels at later passages of the iPSCs, the c-Myc transcript levels were mostly similar in the iPSCs and hESCs, excluding the KTN7 clone which expressed a c-Myc level 2-3 fold higher than of the hESCs. The Klf4 mRNA was considerably higher in two iPSC clones, namely KTN7 and KTR13, exceeding the hESC levels by ˜27-fold and ˜4 fold, respectively, at early passages (5-6). These levels were reduced at later passages (15-24) of clone KTR13, and reached normal hESC levels. In clone KTN7, the Klf4 transcript level was reduced at passage 23, but still exceeded that of the hESCs by ˜20 fold (FIG. 3B). Overall, these findings suggest that the exogenous transgenes are silenced in the majority of HFKT-iPSC clones and that their expression level reaches normal hESC levels.

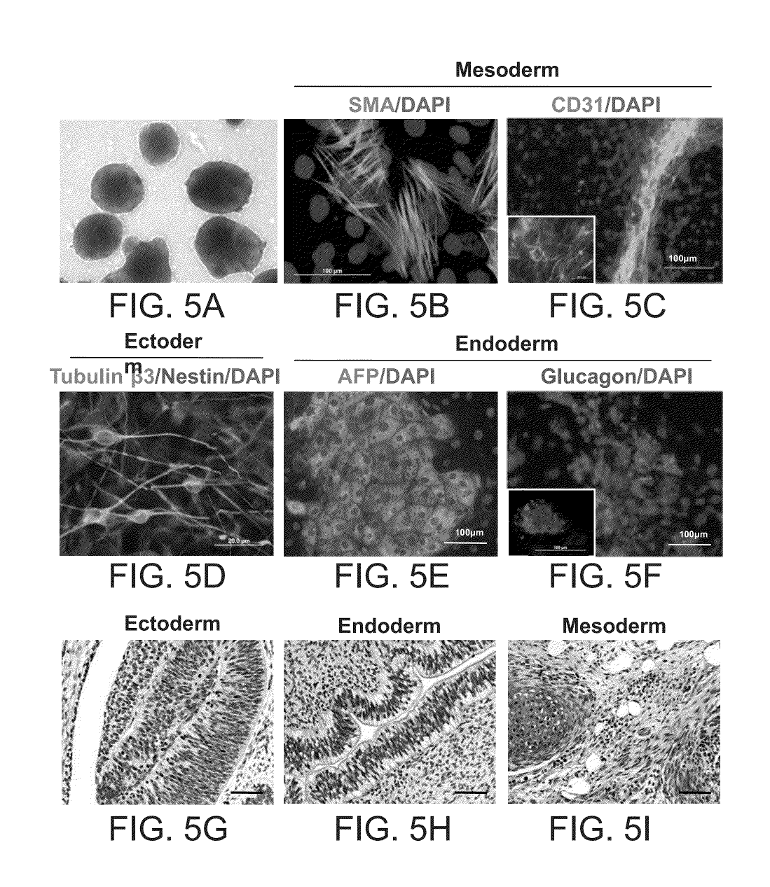

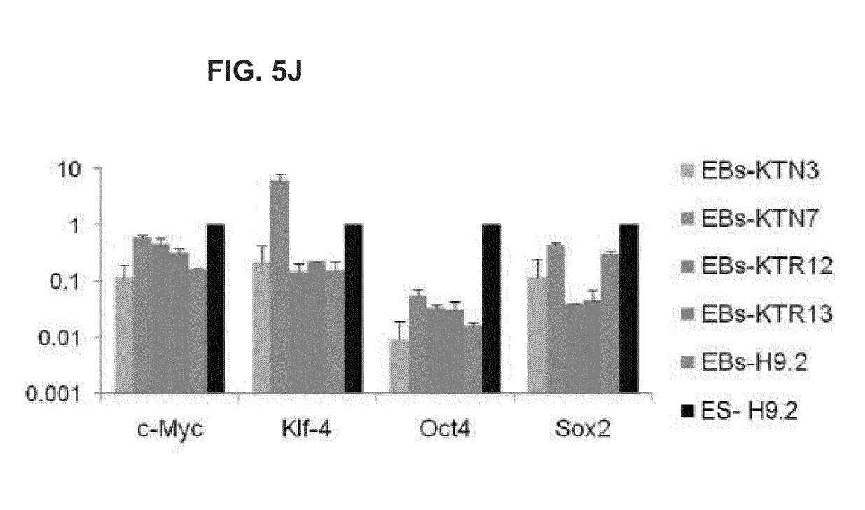

In order to assess the cells' differentiation capacity, differentiation of the four HFKT-iPSC clones was induced in vitro by the generation of embryoid bodies (Ebs) (FIGS. 5A-F), and in vivo using the teratoma assay (FIGS. 5G-I). All the clones were differentiated into derivatives of all three gem layers. EB immunostaining revealed the expression of the mesodermal markers of smooth muscle actin (SMA) and the endothelial marker CD31 (FIGS. 5B,C), the ectodermal tubulin β3 and nestin (FIG. 5D), and the endodermal alpha-fitoprotein (AFP) and glucagon (FIGS. 5E,F). In vivo differentiation of the iPSCs in SCID mice induced the formation of substantial teratomas, containing tissues from all three germ layers: neuronal tissue (FIG. 5F), endodermal epithelium (FIG. 5H) and adipose and muscle tissues (FIG. 5I). Expression levels of the reprogramming factors following HFKT-iPSC differentiation into EBs were also tested. In general, the reduction in transgene levels in the EBs derived from iPSC clones was of 1-2 orders of magnitude, as was the case for the EBs derived from hESCs, and sometimes even greater. Clone KTN7, however, sustained more than a 10-fold higher Klf4 expression following differentiation, relative to H9.2-EBs (FIG. 5J).

### Example 5

### Cardiac Differentiation of HFKT-iPScs

In order to further evaluate the differentiatiability of the HFKT-iPSCs, the present inventors examined their lineage-specific differentiation toward functional cardiomyocytes (CMs). Undifferentiated iPSCs from all four clones were spontaneously differentiated in suspension into EBs and were subsequently placed on gelatin-coated plates. Spontaneously contracting EBs were observed in 3-10% of the total plated EBs from clones KTN3, KTR12 and KTR13 (Table 8).

TABLE 8
Percentage of contracting EBs following spontaneous
differentiation of HFKT-iPSC clones KTR12, KTR13,
KTN3 and KTN7 into EBs aged 14-20 days

| Average, % | | | |
|---|---|---|---|
| contracting EBs | %, Contracting EBs | Passage | Clone |
| 5 | 3 | 14 | KTN3 |
| | 6 | 15 | |
| | 4 | 16 | |
| | 7 | 18 | |
| | 10 | 21 | |
| 0.5 | 0 | 11 | KTN7 |
| | 0 | 14 | |
| | 0 | 15 | |
| | 0 | 21 | |
| | 0 | 22 | |
| | 3.3 | 39 | |
| 3.75 | 0 | 11 | KTR12 |
| | 4 | 13 | |
| | 2.5 | 15 | |
| | 8.5 | 18 | |
| 5.6 | 4.5 | 13 | KTR13 |
| | 8 | 15 | |
| | 4.5 | 18 | |
| | 5.5 | 20 | |

Notably, no contracting EBs from clone KTN7 were found up to passage 39. Immunofluorescence staining of micro-dissected contracting areas (FIG. 6A) demonstrated that the cells expressed cardiac troponin I, α-sarcomeric actinin and myosin heavy chain (MHC), and most importantly, that they exhibited areas of cross-striations. An overlap was found between the fluorescence signal of cardiac troponin, which is a highly cardiac-specific myofilament protein, and each of the other proteins. The functionality of the HFKT-iPSC-CMs was illustrated by robust extracellular electrograms that were recorded by the MEA data acquisition system (FIG. 6B), demonstrating QRS and T-like complexes. Additionally, in order to provide an initial assessment of the functional competence of these cells, we performed whole cell current clamp recordings from isolated dissociated spontaneously contracting areas. These representative recordings (FIG. 6C) demonstrated spontaneously-generated action potentials, with prominent pacemaker potential.

In order to investigate the basic properties of the intracellular Ca²⁺ ([Ca²⁺]ᵢ) handling machinery and the mechanical function, [Ca²⁺]_{I} transients and contractions were recorded from small contracting clusters of the HFKT-iPSC-CMs by means of fura-2 fluorescence and a video edge detector, respectively. The representative traces of [Ca²⁺]ᵢ transients and contractions (FIGS. 6D and 6E, respectively)) from the HFKT-iPSC-CMs (stimulated at 0.6 Hz) were similar to those recorded from the hESC-CMs as well as from the HFF-iPSC-CMs (Germanguz et al. 2009), indicating that these HFKT-iPSC-CMs exhibit ‘cardiac-like’ features of the excitation-contraction coupling machinery. Furthermore, in support of the functionality of the β-adrenergic signaling pathway, isoproterenol caused a marked concentration-dependent positive inotropic effect within 2-3 min, as demonstrated by the increase in [Ca²⁺]ᵢ transient and contraction amplitudes (FIGS. 6D-E). Collectively, these results demonstrate the ability of the HFKT-iPSCs to differentiate into functional CMs.

### Example 6

### Cre-Mediated Excision of a lox-P-Containing STEMCCA Lentiviral Vector

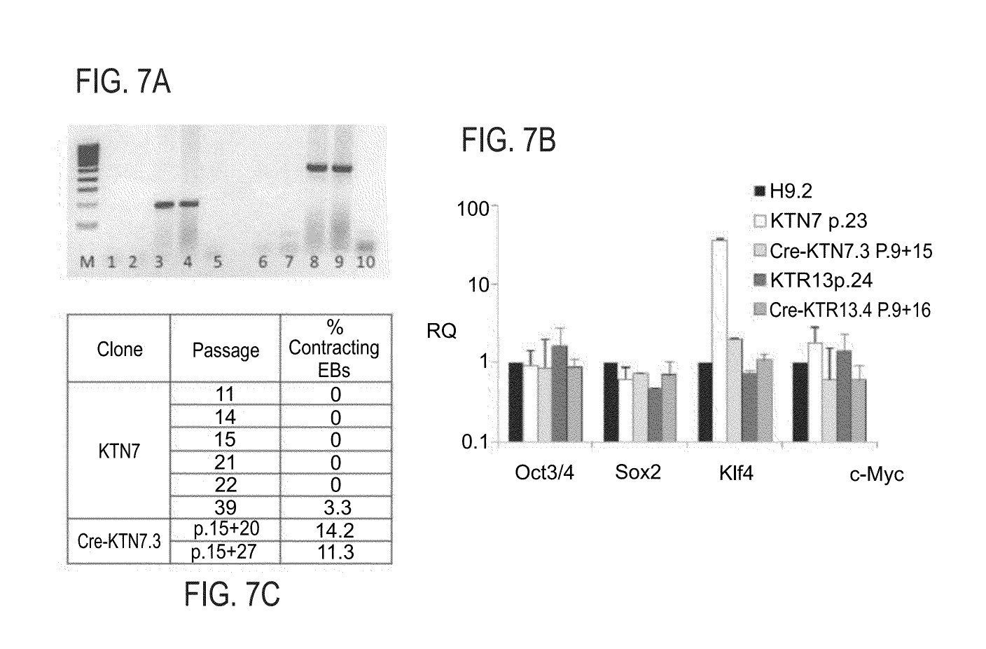

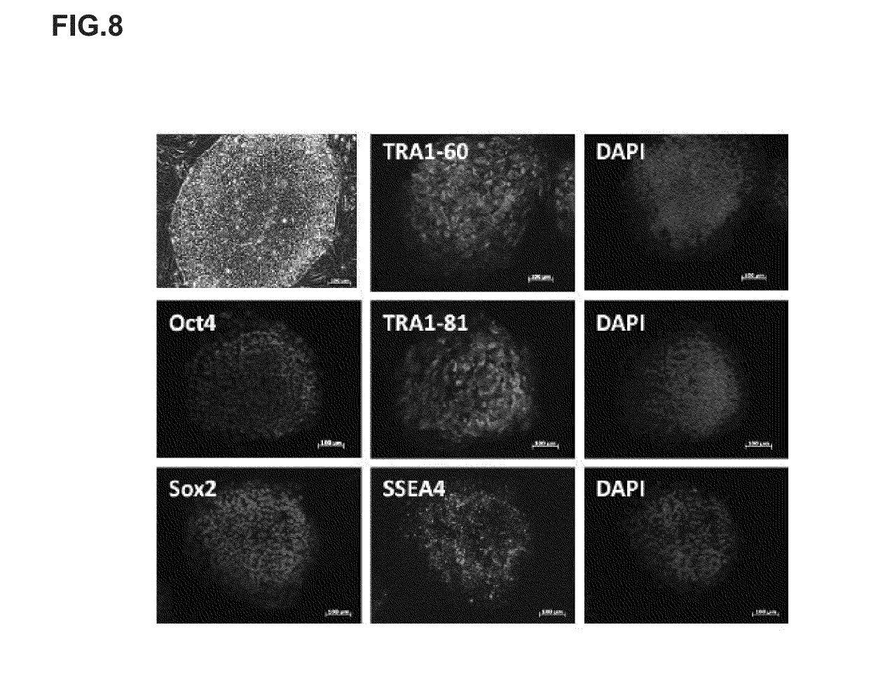

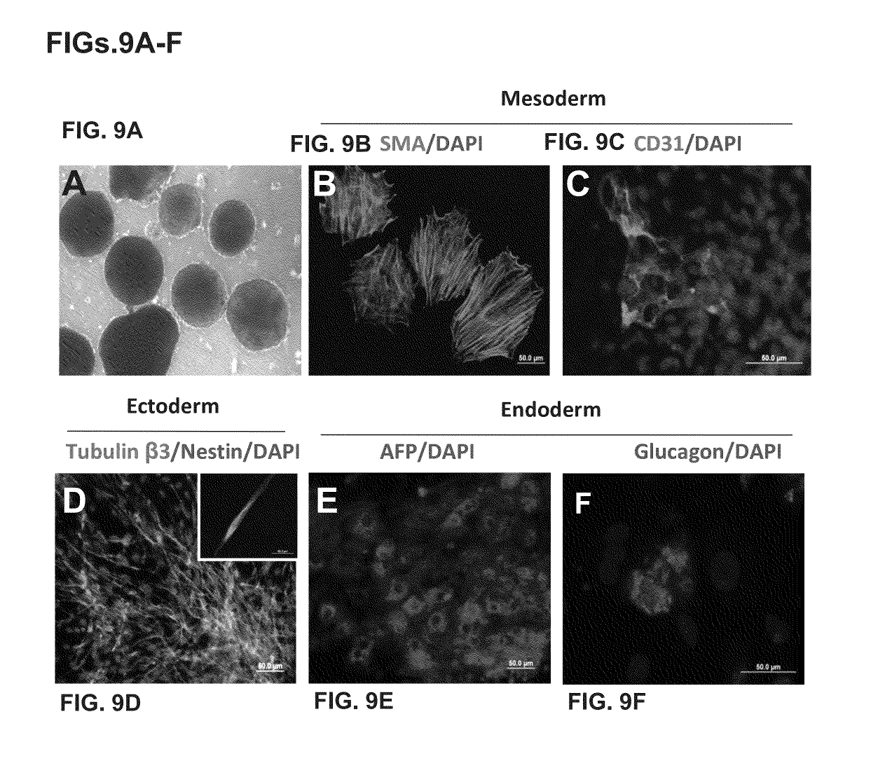

Since the HFKT-iPSCs were generated by excisable lentiviral vector containing lox-P sites flanking the reprogramming transgenes, the present inventors performed Cre-recombinase excision of the human STEMCCA cassette. The excision procedure can efficiently eliminate most of the lentiviral vector including the entire STEMCCA cassette harboring the reprogramming factors. To this end, Puro resistance-Cre-recombinase plasmid was transiently introduced into the two HFKT-iPSC clones KTN7 and KTR13. Following Puromycin selection, small iPSC colonies emerged, which were collected and further expanded in order to analyze the existence of the lentiviral vector. RT-PCR with primers flanking the WPRE sequence of the lentiviral vector showed no positive bends in two HFKT-iPSC clones—Cre-KTN7.3 and Cre-KTR13.4, indicating that the STEMCCA cassette was successfully excised from the cells (FIG. 7A), leaving only small residues of the integrated lentiviral vector. The excised iPSC clones were passaged up to 30 passages post excision, had normal karyotype and resembled hESCs in morphology and the expression of hESC typical markers (FIG. 8). Their pluripotency was demonstrated by the spontaneous differentiation, in vitro, into EBs and the detection of all three germ layers (FIGS. 9A-F). QRT-PCR analysis demonstrated that the reprogramming factors' transcript levels in the undifferentiated Cre-mediated HFKT-iPSC clones were similar to those expressed by the undifferentiated hESCs (FIG. 7B). This was well demonstrated by the Klf4 transcript level in clone KTN7, which was 20-fold higher in the non-excised clone and was reduced to the hESC levels following excision (at clone Cre-KTN7.3). Moreover, the c-Myc transcript levels which were 2-3 fold higher in the original KTN7 and KTR13 clones relative to the hESCs, were reduced even below those of the hESCs following the excision. These results are indicative of the elimination of the undesired transgene expression.

Importantly, the present inventors showed that while KTN7 cells were not able to generate contracting EBs up to passage 39, its excised derivative—clone Cre-KTN7.3, could spontaneously differentiate into contracting EBs with relatively high efficiency (˜13%; FIG. 7C and Table 9). These data demonstrate that elimination of the integrated reprogramming genes is highly desired for the appropriate differentiation into specific lineages.

TABLE 9
Contracting EBs after reprogramming with
excisable or on-excisable vector.

| Clone | Passage | % Contracting EBs |
|---|---|---|
| KTN7 | 11 | 0 |
| | 14 | 0 |
| | 15 | 0 |
| | 21 | 0 |
| | 22 | 0 |
| | 39 | 3.3 |
| Cre-KTN7.3 | p. 15 + 20 | 14.2 |
| | p. 15 + 27 | 11.3 |

### REFERENCES

Aasen, T. and J. C. Belmonte (2010). “Isolation and cultivation of human keratinocytes from skin or plucked hair for the generation of induced pluripotent stem cells.” Nat Protoc 5(2): 371-82.
Aasen, T., A. Raya, M. J. Barrero, E. Garreta, A. Consiglio, F. Gonzalez, R. Vassena, J. Bilic, V. Pekarik, G. Tiscornia, M. Edel, S. Boue and J. C. Izpisua Belmonte (2008). “Efficient and rapid generation of induced pluripotent stem cells from human keratinocytes.” Nat Biotechnol 26(11): 1276-84.
Amit, M., V. Margulets, H. Segev, K. Shariki, I. Laevsky, R. Coleman and J. Itskovitz-Eldor (2003). “Human feeder layers for human embryonic stem cells.” Biol Reprod 68(6): 2150-6.
Brambrink, T., R. Foreman, G. G. Welstead, C. J. Lengner, M. Wernig, H. Suh and R. Jaenisch (2008). “Sequential expression of pluripotency markers during direct reprogramming of mouse somatic cells.” Cell Stem Cell 2(2): 151-9.
Carey, B. W., S. Markoulaki, J. Hanna, K. Saha, Q. Gao, M. Mitalipova and R. Jaenisch (2009). “Reprogramming of murine and human somatic cells using a single polycistronic vector.” Proc Natl Acad Sci USA 106(1): 157-62.
Chang, C. W., Y. S. Lai, K. M. Pawlik, K. Liu, C. W. Sun, C. Li, T. R. Schoeb and T. M. Townes (2009). “Polycistronic lentiviral vector for “hit and run” reprogramming of adult skin fibroblasts to induced pluripotent stem cells.” Stem Cells 27(5): 1042-9.
Chin, M. H., M. J. Mason, W. Xie, S. Volinia, M. Singer, C. Peterson, G. Ambartsumyan, O. Aimiuwu, L. Richter, J. Zhang, I. Khvorostov, V. Ott, M. Grunstein, N. Lavon, N. Benvenisty, C. M. Croce, A. T. Clark, T. Baxter, A. D. Pyle, M. A. Teitell, M. Pelegrini, K. Plath and W. E. Lowry (2009). “Induced pluripotent stem cells and embryonic stem cells are distinguished by gene expression signatures.” Cell Stem Cell 5(1): 111-23.
Dolnikov, K., M. Shilkrut, N. Zeevi-Levin, S. Gerecht-Nir, M. Amit, A. Danon, J. Itskovitz-Eldor and O. Binah (2006). “Functional properties of human embryonic stem cell-derived cardiomyocytes: intracellular Ca2+ handling and the role of sarcoplasmic reticulum in the contraction.” Stem Cells 24(2): 236-45.
Feng, Jia-Hui Ng, Jian-Chien Dominic Heng, and Huck-Hui Ng. (2009) Molecules that Promote or Enhance Reprogramming of Somatic Cells to Induced Pluripotent Stem Cells. Cell Stem Cell 4: 301-312
Germanguz, I., O. Sedan, N. Zeevi-Levin, R. Shtreichman, E. Barak, A. Ziskind, S. Eliyahu, G. Meiry, M. Amit, J. Itskovitz-Eldor and O. Binah (2009). “Molecular characterization and functional properties of cardiomyocytes derived from human inducible pluripotent stem cells.” J Cell Mol Med.
Haase, A., R. Olmer, K. Schwanke, S. Wunderlich, S. Merkert, C. Hess, R. Zweigerdt, I. Gruh, J. Meyer, S. Wagner, L. S. Maier, D. W. Han, S. Glage, K. Miller, P. Fischer, H. R. Scholer and U. Martin (2009). “Generation of induced pluripotent stem cells from human cord blood.” Cell Stem Cell 5(4): 434-41.
Hochedlinger, K. and K. Plath (2009). “Epigenetic reprogramming and induced pluripotency.” Development 136(4): 509-23.
Hochedlinger, K., Y. Yamada, C. Beard and R. Jaenisch (2005). “Ectopic expression of Oct-4 blocks progenitor-cell differentiation and causes dysplasia in epithelial tissues.” Cell 121(3): 465-77.
Huangfu, D., K. Osafune, R. Maehr, W. Guo, A. Eijkelenboom, S. Chen, W. Muhlestein and D. A. Melton (2008b). “Induction of pluripotent stem cells from primary human fibroblasts with only Oct4 and Sox2.” Nat Biotechnol 26(11): 1269-75.
Itskovitz-Eldor, J., M. Schuldiner, D. Karsenti, A. Eden, O. Yanuka, M. Amit, H. Soreq and N. Benvenisty (2000). “Differentiation of human embryonic stem cells into embryoid bodies compromising the three embryonic germ layers.” Mol Med 6(2): 88-95.
Kim, J. B., V. Sebastiano, G. Wu, M. J. Arauzo-Bravo, P. Sasse, L. Gentile, K. Ko, D. Ruau, M. Ehrich, D. van den Boom, J. Meyer, K. Hubner, C. Bernemann, C. Ortmeier, M. Zenke, B. K. Fleischmann, H. Zaehres and H. R. Scholer (2009). “Oct4-induced pluripotency in adult neural stem cells.” Cell 136(3): 411-9.
Kim, J. B., H. Zaehres, G. Wu, L. Gentile, K. Ko, V. Sebastiano, M. J. Arauzo-Bravo, D. Ruau, D. W. Han, M. Zenke and H. R. Scholer (2008). “Pluripotent stem cells induced from adult neural stem cells by reprogramming with two factors.” Nature 454(7204): 646-50.
Kiskinis, E. and K. Eggan (2010). “Progress toward the clinical application of patient-specific pluripotent stem cells.” J Clin Invest 120(1): 51-9.
Limat, A. and F. K. Noser (1986). “Serial cultivation of single keratinocytes from the outer root sheath of human scalp hair follicles.” J Invest Dermatol 87(4): 485-8.
Lowry, W. E., L. Richter, R. Yachechko, A. D. Pyle, J. Tchieu, R. Sridharan, A. T. Clark and K. Plath (2008). “Generation of human induced pluripotent stem cells from dermal fibroblasts.” Proc Natl Acad Sci USA 105(8): 2883-8.
Marchetto, M. C., G. W. Yeo, O. Kainohana, M. Marsala, F. H. Gage and A. R. Muotri (2009). “Transcriptional signature and memory retention of human-induced pluripotent stem cells.” PLoS One 4(9): e7076.
Meiry, G., Y. Reisner, Y. Feld, S. Goldberg, M. Rosen, N. Ziv and O. Binah (2001). “Evolution of action potential propagation and repolarization in cultured neonatal rat ventricular myocytes.” J Cardiovasc Electrophysiol 12(11): 1269-77.
Mostoslaysky, G., A. J. Fabian, S. Rooney, F. W. Alt and R. C. Mulligan (2006). “Complete correction of murine Artemis immunodeficiency by lentiviral vector-mediated gene transfer.” Proc Natl Acad Sci USA 103(44): 16406-11.
Nakagawa, M., M. Koyanagi, K. Tanabe, K. Takahashi, T. Ichisaka, T. Aoi, K. Okita, Y. Mochiduki, N. Takizawa and S. Yamanaka (2008). “Generation of induced pluripotent stem cells without Myc from mouse and human fibroblasts.” Nat Biotechnol 26(1): 101-6.
Okita, K., T. Ichisaka and S. Yamanaka (2007). “Generation of germline-competent induced pluripotent stem cells.” Nature 448(7151): 313-7.
Park, I. H., P. H. Lerou, R. Zhao, H. Huo and G. Q. Daley (2008). “Generation of human-induced pluripotent stem cells.” Nat Protoc 3(7): 1180-6.
Pellegrini, Elena Dellambra, Osvaldo Golisano, Enrica Martinelli, Ivana Fantozzi, Sergio Bondanza (2001). Proceedings of the National Academy of Sciences 98: 3156-3161.
Reisner, Y., G. Meiry, N. Zeevi-Levin, D. Y. Barac, I. Reiter, Z. Abassi, N. Ziv, S. Kostin, J. Schaper, M. R. Rosen and O. Binah (2009). “Impulse conduction and gap junctional remodelling by endothelin-1 in cultured neonatal rat ventricular myocytes.” J Cell Mol Med 13(3): 562-73.
Rogers, G. E. (2004). “Hair follicle differentiation and regulation.” Int J Dev Biol 48(2-3): 163-70.
Schneider, M. R., R. Schmidt-Ullrich and R. Paus (2009). “The hair follicle as a dynamic miniorgan.” Curr Biol 19(3): R132-42.
Sedan, O., K. Dolnikov, N. Zeevi-Levin, N. Leibovich, M. Amit, J. Itskovitz-Eldor and O. Binah (2008). “1,4,5-Inositol trisphosphate-operated intracellular Ca(2+) stores and angiotensin-II/endothelin-1 signaling pathway are functional in human embryonic stem cell-derived cardiomyocytes.” Stem Cells 26(12): 3130-8.
Soldner, F., D. Hockemeyer, C. Beard, Q. Gao, G. W. Bell, E. G. Cook, G. Hargus, A. Blak, O. Cooper, M. Mitalipova, O. Isacson and R. Jaenisch (2009). “Parkinson's disease patient-derived induced pluripotent stem cells free of viral reprogramming factors.” Cell 136(5): 964-77.
Sommer, C. A., A. G. Sommer, T. A. Longmire, C. Christodoulou, D. D. Thomas, M. Gostissa, F. W. Alt, G. J. Murphy, D. N. Kotton and G. Mostoslaysky (2010). “Excision of reprogramming transgenes improves the differentiation potential of iPS cells generated with a single excisable vector.” Stem Cells 28(1): 64-74.
Sommer, C. A., M. Stadtfeld, G. J. Murphy, K. Hochedlinger, D. N. Kotton and G. Mostoslaysky (2009). “Induced pluripotent stem cell generation using a single lentiviral stem cell cassette.” Stem Cells 27(3): 543-9.
Takahashi, K., K. Tanabe, M. Ohnuki, M. Narita, T. Ichisaka, K. Tomoda and S. Yamanaka (2007). “Induction of pluripotent stem cells from adult human fibroblasts by defined factors.” Cell 131(5): 861-72.
Thomson, J. A., J. Itskovitz-Eldor, S. S. Shapiro, M. A. Waknitz, J. J. Swiergiel, V. S. Marshall and J. M. Jones (1998). “Embryonic stem cell lines derived from human blastocysts.” Science 282(5391): 1145-7.
Yang, A., R. Schweitzer, D. Sun, M. Kaghad, N. Walker, R. T. Bronson, C. Tabin, A. Sharpe, D. Caput, C. Crum and F. McKeon (1999). “p63 is essential for regenerative proliferation in limb, craniofacial and epithelial development.” Nature 398(6729): 714-8.
Yu, J., M. A. Vodyanik, K. Smuga-Otto, J. Antosiewicz-Bourget, J. L. Frane, S. Tian, J. Nie, G. A. Jonsdottir, V. Ruotti, R. Stewart, Slukvin, II and J. A. Thomson (2007). “Induced pluripotent stem cell lines derived from human somatic cells.” Science 318(5858): 1917-20.
Zhang, J., G. F. Wilson, A. G. Soerens, C. H. Koonce, J. Yu, S. P. Palecek, J. A. Thomson and T. J. Kamp (2009). “Functional cardiomyocytes derived from human induced pluripotent stem cells.” Circ Res 104(4): e30-41.

1. A method for generating induced pluripotent stem (iPS) cells from isolated hair follicles, the method comprising:
a. culturing isolated hair follicle keratinocytes on a layer of feeder cells, wherein said isolated keratinocytes are generated by dissociating cells of the hair follicle, so as to generate colonies of hair follicle keratinocytes;
b. detaching said colonies of hair follicle keratinocytes from said feeder cells so as to generate detached keratinocytes;
c. infecting said detached keratinocytes with a virus comprising a nucleic acid molecule encoding at least OCT4 and SOX2 and dedifferentiation factors so as to generate infected keratinocytes; and
d. culturing said infected keratinocytes on a layer of feeder cells in a culture medium until iPS cells are formed, thereby generating iPS cells.

2. The method of claim 1, wherein said nucleic acid molecule further encodes KLF4 and/or C-MYC.

3. The method of claim 1, wherein said colonies comprise between 20-30 hair follicle keratinocytes.

4. The method of claim 1, wherein said isolated hair follicle keratinocytes are in contact with said virus for less than 2 hours.

5. The method of claim 1, wherein said isolated hair follicle keratinocytes are in contact with said virus for less than one hour.

6. The method of claim 1, wherein said virus is a lentivirus.

7. The method of claim 1, wherein said isolated hair follicle keratinocytes are not passaged for more than 3 passages.

8. The method of claim 1, wherein said isolated hair follicle keratinocytes are passaged for 2-3 passages.

9. The method of claim 1, wherein said dissociating is effected using trypsin.

10. The method of claim 1, wherein said infecting is effected during centrifugation at a centrifugal force of about 200 g to about 1000 g.

11. The method of claim 1, wherein said infecting is effected at a temperature between 25° C.-37° C.

12. The method of claim 1, wherein said feeder cells comprise 3T3 cells or mouse embryonic feeder (MEF) cells.

13. The method of claim 1, wherein said nucleic acid molecule further encodes LoxP sites.

14. The method of claim 13, further comprising excising said nucleic acid molecule following step (d) by contacting said iPS cells with a cre-recombinase enzyme.

15. The method of claim 2, wherein said at least one dedifferentiation factor further comprises Nanog and/or Lin 28.

16-19. (canceled)

20. The method of claim 1, wherein said nucleic acid molecule comprises a sequence as set forth in SEQ ID NO: 1.

21. The method of claim 1, wherein, for at least a portion of a time of said culturing said infected keratinocytes, said culture medium comprises a small molecule.

22. The method of claim 21, wherein said small molecule is selected from the group consisting of a glycogen synthase kinase 3 (GSK-3) inhibitor, a lysine-specific demethylaseinhibitor, a histone methyltransferase inhibitor, a histone deacetylase inhibitor, a TGF-β inhibitor; a combination of inhibitors of mitogen-activated protein kinase (MAPK/ERK kinase or MEK) and GSK-3; and an L-type calcium channel agonist.

23. The method of claim 22, wherein said GSK-3 inhibitor comprises CHIR99021.

24. The method of claim 22, wherein said lysine-specific demethylase inhibitor is Parnate (Tranylcypromine).

25. The method of claim 1, wherein said detaching is effected using EDTA.

26. Induced pluripotent stem (iPS) cells obtained according to the method of claim 1.

27. A cell line of the iPS cells of claim 26.

28. The iPS cells of claim 26, for use in tissue regeneration.

29. The iPS cells of claim 28, wherein said tissue regeneration is cardiac tissue regeneration.

30. A pharmaceutical composition comprising the iPS cells of claim 26.

31. A method of generating lineage specific cells, the method comprising:
(a) generating iPS cells according to the method of claim 1; and
(b) ex vivo differentiating said iPS cells into lineage specific cells, thereby generating said lineage specific cells.


SEQUENCE LISTING (36 sequences)

SEQ ID NO: 1 - DNA, 12535 bp, Artificial sequence
  Full sequence of the human single polycistronic lentiviral vector - STEMCCA cassette
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactcgggttcgggtgcgccagtaaagcagacattaaactttgatttgctgaaacttgcaggtgatgtagagtcaaatccaggtccaatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatgatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtccggaagcggagccactaacttctccctgttgaaacaagcaggggatgtcgaagagaatcccgggccaatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaagtcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgcaggtaccataacttcgtataatgtatgctatacgaagttatggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 2 - DNA, 11926 bp, Artificial sequence
  Full sequence of the human single polycistronic lentiviral vector - Cherry cassette
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactcgggttcgggtgcgccagtaaagcagacattaaactttgatttgctgaaacttgcaggtgatgtagagtcaaatccaggtccaatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatgatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtccggaagcggagccactaacttctccctgttgaaacaagcaggggatgtcgaagagaatcccgggccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgcaggtaccataacttcgtataatgtatgctatacgaagttatggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 3 - DNA, 19 bp, Artificial sequence
  Single strand DNA oligonucleotide
ccacatcgctcagaaccat

SEQ ID NO: 4 - DNA, 22 bp, Artificial sequence
  Single strand DNA oligonucleotide
ggcaacaatatccatttaccag

SEQ ID NO: 5 - DNA, 19 bp, Artificial sequence
  Single strand DNA oligonucleotide
ctcaccctgggggttctat

SEQ ID NO: 6 - DNA, 21 bp, Artificial sequence
  Single strand DNA oligonucleotide
ctccaggttgcctctctcact

SEQ ID NO: 7 - DNA, 14 bp, Artificial sequence
  Single strand DNA oligonucleotide
tgagtgtggatcca

SEQ ID NO: 8 - DNA, 14 bp, Artificial sequence
  Single strand DNA oligonucleotide
tgaataagcagatc

SEQ ID NO: 9 - DNA, 15 bp, Artificial sequence
  Single strand DNA oligonucleotide
ctcaaggcacacctg

SEQ ID NO: 10 - DNA, 15 bp, Artificial sequence
  Single strand DNA oligonucleotide
agtgcctggtcagtt

SEQ ID NO: 11 - DNA, 18 bp, Artificial sequence
  Single strand DNA oligonucleotide
actctgaggaggaacaag

SEQ ID NO: 12 - DNA, 19 bp, Artificial sequence
  Single strand DNA oligonucleotide
tggagacgtggcacctctt

SEQ ID NO: 13 - DNA, 14 bp, Artificial sequence
  Single strand DNA oligonucleotide
gggaggggtgcaaa

SEQ ID NO: 14 - DNA, 15 bp, Artificial sequence
  Single strand DNA oligonucleotide
cacagcaaatgacag

SEQ ID NO: 15 - DNA, 14 bp, Artificial sequence
  Single strand DNA oligonucleotide
acagtccagcaggt

SEQ ID NO: 16 - DNA, 15 bp, Artificial sequence
  Single strand DNA oligonucleotide
cttgtctttgcccgt

SEQ ID NO: 17 - DNA, 20 bp, Artificial sequence
  Single strand DNA oligonucleotide
gaccattgaggacctgagga

SEQ ID NO: 18 - DNA, 20 bp, Artificial sequence
  Single strand DNA oligonucleotide
catacttggtgcggaagtca

SEQ ID NO: 19 - DNA, 18 bp, Artificial sequence
  Single strand DNA oligonucleotide
tttcccaccccgagatga

SEQ ID NO: 20 - DNA, 16 bp, Artificial sequence
  Single strand DNA oligonucleotide
tgcggcgagcatccat

SEQ ID NO: 21 - DNA, 20 bp, Artificial sequence
  Single strand DNA oligonucleotide
gctaacaaaaacccacatct

SEQ ID NO: 22 - DNA, 20 bp, Artificial sequence
  Single strand DNA oligonucleotide
gctaacaaaaacccacatct

SEQ ID NO: 23 - DNA, 16 bp, Artificial sequence
  Single strand DNA oligonucleotide
ggctcctgaacctggg

SEQ ID NO: 24 - DNA, 18 bp, Artificial sequence
  Single strand DNA oligonucleotide
agctttggctgaccttcc

SEQ ID NO: 25 - DNA, 18 bp, Artificial sequence
  Single strand DNA oligonucleotide
tgtgataaggaccaaggc

SEQ ID NO: 26 - DNA, 19 bp, Artificial sequence
  Single strand DNA oligonucleotide
gagcaaattgcagaatgag

SEQ ID NO: 27 - DNA, 23 bp, Artificial sequence
  Single strand DNA oligonucleotide
actcactctagtgataaatcggg

SEQ ID NO: 28 - DNA, 20 bp, Artificial sequence
  Single strand DNA oligonucleotide
ggaaccagaaactgtggcat

SEQ ID NO: 29 - DNA, 20 bp, Artificial sequence
  Single strand DNA oligonucleotide
catgtgctctgcaccataag

SEQ ID NO: 30 - DNA, 20 bp, Artificial sequence
  Single strand DNA oligonucleotide
ggaaccagaaactgtggcat

SEQ ID NO: 31 - DNA, 18 bp, Artificial sequence
  Single strand DNA oligonucleotide
aatctgttctggcaatgg

SEQ ID NO: 32 - DNA, 22 bp, Artificial sequence
  Single strand DNA oligonucleotide
ttatgtttactttctcagtggg

SEQ ID NO: 33 - DNA, 22 bp, Artificial sequence
  Single strand DNA oligonucleotide
cagaaaatagttcagaccacca

SEQ ID NO: 34 - DNA, 21 bp, Artificial sequence
  Single strand DNA oligonucleotide
gggacaagagaaagttgaaca

SEQ ID NO: 35 - DNA, 20 bp, Artificial sequence
  Single strand DNA oligonucleotide
tggactataggacccccttc

SEQ ID NO: 36 - DNA, 20 bp, Artificial sequence
  Single strand DNA oligonucleotide
gagaacagcctgtcacacct